(19) United States
(12) Patent Application Publication
(10) Pub. No.: US 2013/0018214 A1
(43) Pub. Date: Jan. 17, 2013

(54) CATALYST COMPOSITION FOR OLIGOMERIZATION OF ETHYLENE AND PROCESSES OF OLIGOMERIZATION

(71) Applicants: Mingfang Zheng, Beijing (CN); Weizhen Li, Beijing (CN); Huaijie Wang, Beijing (CN); Jun Liu, Beijing (CN); Haiying Zhang, Beijing (CN); Yu Zhou, Beijing (CN); Tonglin Li, Beijing (CN); Lan Zhao, Beijing (CN); Jilong Wang, Beijing (CN); Hongfei Wu, Beijing (CN); Yuling Piao, Beijing (CN); Junlong Sui, Beijing (CN)

(72) Inventors: Mingfang Zheng, Beijing (CN); Weizhen Li, Beijing (CN); Huaijie Wang, Beijing (CN); Jun Liu, Beijing (CN); Haiying Zhang, Beijing (CN); Yu Zhou, Beijing (CN); Tonglin Li, Beijing (CN); Lan Zhao, Beijing (CN); Jilong Wang, Beijing (CN); Hongfei Wu, Beijing (CN); Yuling Piao, Beijing (CN); Junlong Sui, Beijing (CN)

(73) Assignee: China Petroleum & Chemical Corporation, Beijing (CN)

(21) Appl. No.: 13/637,976

(22) PCT Filed: Mar. 30, 2011
(86) PCT No.: PCT/CN11/00550

§ 371 Date: Sep. 27, 2012

(30) Foreign Application Priority Data
Mar. 31, 2010 (CN) 201010138127.1
Sep. 29, 2010 (CN) 201010500316.9

(51) Int. Cl. B01J 31/12; C07C 2/26

(52) U.S. Cl. 585/513; 502/167

(57) ABSTRACT

The present invention provides a catalyst composition for the ethylene oligomerization, which comprises 2-imino-1,10-phenanthroline coordinated iron (II), cobalt (II) or nickel (II) chloride as main catalyst and triethylaluminum as cocatalyst. The present invention also provides a process for oligomerization of ethylene is provided, wherein a catalyst composition comprising 2-imino-1,10-phenanthroline coordinated iron (II), cobalt (II) or nickel (II) chloride as main catalyst and triethylaluminum as cocatalyst is used, and the molar ratio of aluminum in the cocatalyst to central metal in the main catalyst ranges from 30 to less than 200. According to the present invention, another process for oligomerization of ethylene is also provided, wherein a catalyst composition comprising 2-imino-1,10-phenanthroline coordinated iron (II), cobalt (II) or nickel (II) chloride as main catalyst and triethylaluminum as cocatalyst is used, and the temperature of ethylene oligomerization ranges from −10 to 19° C. According to the present invention, the price of cocatalyst i.e. triethylaluminum, is low, just a fraction of that of methylaluminoxane, the amount of cocatalyst is therefore significantly reduced, with the catalytic activity is still acceptable, thus the cost of ethylene oligomerization is significantly reduced. In view of both the catalytic activity and the cost, the present invention is highly applicable in industry.

### CROSS REFERENCE TO RELATED APPLICATIONS

This is a national stage entry based on International Application No. PCT/CN2011/000550, which in turn claims priority to Chinese Patent Application No. CN 201010138127.1 filed on Mar. 31, 2010 and to No. CN 201010500316.9 filed on Sep. 29, 2010, each of which is hereby incorporated by reference in its entirety.

### TECHNICAL FIELD

The present invention relates to the field of ethylene oligomerization, and more specifically to a catalyst composition of 2-imino-1,10-phenanthroline coordinated iron (II), cobalt (II) or nickel (II) chloride and triethylaluminum. The invention also relates to processes for ethylene oligomerization in the presence of the above-mentioned catalyst composition.

### BACKGROUND

Linear alpha olefins (LAOS) are widely used in various applications, such as ethylene co-monomers, intermediates in production of surfactants, plasticizer alcohols, synthetic lubricants and oil additives, etc. Recently, with the development of polyolefin industry, the worldwide demand for alpha olefins grows rapidly. Currently, most of alpha olefins are prepared based on ethylene oligomerization. The common catalysts used in the ethylene oligomerization mainly include nickel-, chromium-, zirconium-, and alumina-based catalyst systems, and so on. Recently, the complex of iron (II) and cobalt (II) with imino-pyridyl tridentate ligands for catalyzing ethylene oligomerization have been reported respectively by Brookhart's group (see Brookhart M et al, J. Am. Chem. Soc., 1998, 120, 7143-7144 and WO99/02472) and Gibson's group (see Gibson V. C. et al, Chem. Commun., 1998, 849-850 and Chem. Eur. J., 2000, 2221-2231), in which both the catalytic activity and selectivity of alpha olefins are high.

A catalyst for ethylene oligomerization and polymerization is disclosed in CN1850339A filed by ICCAS (Institute of Chemistry, Chinese Academy of Sciences), which is 2-imino-1,10-phenanthroline coordinated iron (II), cobalt (II) or nickel (II) chloride. In the presence of methylaluminoxane as cocatalyst, the above-mentioned catalyst as the main catalyst has a good catalytic activity for ethylene oligomerization and polymerization, wherein the iron complex shows a high catalytic activity for ethylene oligomerization and polymerization, the oligomerization activity is the highest at a reaction temperature of 40° C., and the oligomerization and polymerization activity are obviously enhanced with the increase of pressure. The oligomerization products include C₄ olefin, C₆ olefins, C₈ olefins, C₁₀ olefins, C₁₂ olefins, C₁₄ olefins, C₁₆ olefins, C₁₈ olefins, C₂₀ olefins, C₂₂ olefins and so on, and the polymerization products are low molecular weight polyolefin and waxy polyolefin. CN1850339A also discloses that, when triethylaluminum is used as the cocatalyst and 2-acetyl-1,10-phenanthroline (2,6-diethylanil) FeCl₂ is used as the main catalyst, Al/Fe equals to 500, the reaction temperature is 40° C., the reaction pressure is 1 MPa and the reaction time lasts 1 h, the oligomerization activity will be 2.71×10⁵. It further discloses that, when triisobutylalumium and diethylalumium chloride are used as cocatalysts, the oligomerization activity is low even with a high amount of cocatalysts (Al/Fe=500).

It can be seen from the teachings of the above-mentioned patent that, when triethylaluminum is used as cocatalyst, the oligomerization activity is still low even with a high amount of cocatalyst, which leads to a poor practicability. Therefore, costly methylaluminoxane is used as cocatalyst in the patent. However, the high amount and high cost of methylaluminoxane will definitely lead to a high production cost when methylaluminoxane is used as the cocatalyst in ethylene oligomerization in a large-scale manner.

Additionally, publication “Iron Complexes Bearing 2-Imino-1,10-phenanthrolinyl Ligands as Highly Active Catalysts for Ethylene Oligomerization” (see Sun wenhua et. al., Journal of Organometallics 25 (2006) 666-677) discloses in Table 2 thereof that, when 2-acetyl-1,10-phenanthroline (2,6-diethylanil)FeCl₂ is used as main catalyst for ethylene oligomerization, the ethylene oligomerization activity will not increase or decrease monotonically as the reaction temperature changes; instead, the oligomerization activity increases with the increase of temperature when the reaction temperature is within the range of 20 to 40° C., but decreases with the increase of temperature when the reaction temperature is within the range of 40 to 60° C. The result is further confirmed in Table 4 of another literature by the same author in Journal of Organometallics 26 (2007) 2720-2734, in which diethylalumium chloride is used as cocatalyst for ethylene oligomerization.

### SUMMARY OF THE INVENTION

It is therefore an object of the present invention to provide a low cost catalyst composition and a process for ethylene oligomerization, which can overcome or at least partly eliminate the defects existing in the prior arts, so that they can be used in the large-scale industrial applications. Surprisingly, it is found that when a catalyst composition comprising a small amount of triethylaluminum as cocataylst and 2-imino-1,10-phenanthroline coordinated iron (II), cobalt (II) or nickel (II) chloride as main catalyst is used for ethylene oligomerization, the catalytic activity is acceptable, which is significantly different from the low activity assumed in the prior arts. Due to the low price and low amount of triethylaluminum and the acceptable catalytic activity, the catalyst composition can be satisfactorily used in the ethylene oligomerization process in the large-scale industrial applications.

According to an aspect of the present invention, a catalyst composition for ethylene oligomerization is provided, comprising 2-imino-1,10-phenanthroline coordinated iron (II), cobalt (II) or nickel (II) chloride as shown in Formula (I) as main catalyst and triethylaluminum as cocatalyst, wherein the molar ratio of aluminum in the cocatalyst to central metal in the main catalyst ranges from 30 to less than 200:

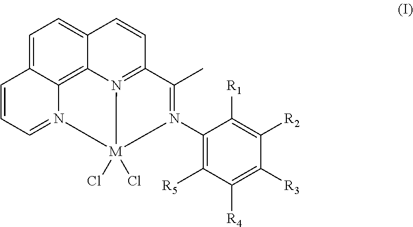

wherein M is the central metal selected from Fe²⁺, Co²⁺ and Ni²⁺; R₁-R₅ are independently selected from hydrogen, (C₁-C₆) alkyl, halogen, (C₁-C₆) alkoxyl and nitro group.

In the present invention, the term “(C₁-C₆) alkyl group” refers to saturated straight chain or branched chain alkyl group with 1-6 carbon atoms. Said (C₁-C₆) alkyl group includes methyl, ethyl, n-propyl, isopropyl, n-butyl, isobutyl, sec-butyl, tert-butyl, n-pentyl, sec-pentyl, n-hexyl and sec-hexyl, preferably methyl, ethyl or isopropyl.

In the present invention, the term “(C₁-C₆) alkoxyl group” refers to the group obtained from the bond of (C₁-C₆) alkyl group linked with an Oxygen atom. Said (C₁-C₆) alkoxyl group includes methoxyl, ethoxyl, n-propoxyl, isopropoxyl, n-butoxyl, isobutoxyl, sec-butoxyl, tert-butoxyl, n-pentoxyl, sec-pentoxyl, n-hexyloxyl and sec-hexyloxyl, preferably methoxyl or ethoxyl.

In the present invention, the term “halogen” includes F, Cl, Br and I, preferably F, Cl or Br.

In an advantageous embodiment of said catalyst composition, the molar ratio of aluminum in the cocatalyst to central metal (i.e. Fe²⁺, Co²⁺ or Ni²⁺) in the main catalyst ranges from 50 to less than 200, preferably from 100 to 199.8, more preferably from 148 to 196, most preferably from 178 to 196.

In another advantageous embodiment of said catalyst composition, M and R₁-R₅ in the main catalyst are defined as follows:
1: M=Fe²⁺, R₁=Me, R₂═R₃═R₄═R₅═H;
2: M=Fe²⁺, R₂=Me, R₁═R₃═R₄═R₅═H;
3: M=Fe²⁺, R₃=Me, R₁═R₂═R₄═R₅═H;
4: M=Fe²⁺, R₁═R₂=Me, R₃═R₄═R₅═H;
5: M=Fe²⁺, R₁═R₃=Me, R₂═R₄═R₅═H;
6: M=Fe²⁺, R₁═R₄=Me, R₂═R₃═R₅═H;
7: M=Fe²⁺, R₁═R₅=Me, R₂═R₃═R₄═H;
8: M=Fe²⁺, R₂═R₃=Me, R₁═R₄═R₅═H;
9: M=Fe²⁺, R₂═R₄=Me, R₁═R₃═R₅═H;
10: M=Fe²⁺, R₁═R₃═R₅=Me, R₂═R₄═H;
11: M=Fe²⁺, R₁=Et, R₂═R₃═R₄═R₅═H;
12: M=Fe²⁺, R₁=Et, R₅=Me, R₂═R₃═R₄═H;
13: M=Fe²⁺, R₁═R₅=Et, R₂═R₃═R₄═H;
14: M=Fe²⁺, R₁=iPr, R₂═R₃═R₄═R₅═H;
15: M=Fe²⁺, R₁═R₅=iPr, R₂═R₃═R₄═H;
16: M=Co²⁺, R₁=Me, R₂═R₃═R₄═R₅═H;
17: M=Co²⁺, R₂=Me, R₁═R₃═R₄═R₅═H;
18: M=Co²⁺, R₃=Me, R₁═R₂═R₄═R₅═H;
19: M=Co²⁺, R₁═R₂=Me, R₃═R₄═R₅═H;
20: M=Co²⁺, R₁═R₃=Me, R₂═R₄═R₅═H;
21: M=Co²⁺, R₁═R₄=Me, R₂═R₃═R₅═H;
22: M=Co²⁺, R₁═R₅=Me, R₂═R₃═R₄═H;
23: M=Co²⁺, R₂═R₃=Me, R₁═R₄═R₅═H;
24: M=Co²⁺, R₂═R₄=Me, R₁═R₃═R₅═H;
25: M=Co²⁺, R₁═R₃═R₅=Me, R₂═R₄═H;
26: M=Co²⁺, R₁=Et, R₂═R₃═R₄═R₅═H;
27: M=Co²⁺, R₁=Et, R₅=Me, R₂═R₃═R₄═H;
28: M=Co²⁺, R₁═R₅=Et, R₂═R₃═R₄═H;
29: M=Co²⁺, R₁=iPr, R₂═R₃═R₄═R₅═H;
30: M=Co²⁺, R₁═R₅=iPr, R₂═R₃═R₄═H;
31: M=Ni²⁺, R₁=Me, R₂═R₃═R₄═R₅═H;
32: M=Ni²⁺, R₂=Me, R₁═R₃═R₄═R₅═H;
33: M=Ni²⁺, R₃=Me, R₁═R₂═R₄═R₅═H;
34: M=Ni²⁺, R₁═R₂=Me, R₃═R₄═R₅═H;
35: M=Ni²⁺, R₁═R₃=Me, R₂═R₄═R₅═H;
36: M=Ni²⁺, R₁═R₄=Me, R₂═R₃═R₅═H;
37: M=Ni²⁺, R₁═R₅=Me, R₂═R₃═R₄═H;
38: M=Ni²⁺, R₂═R₃=Me, R₁═R₄═R₅═H;
39: M=Ni²⁺, R₂═R₄=Me, R₁═R₃═R₅═H;
40: M=Ni²⁺, R₁═R₃═R₅=Me, R₂═R₄═H;
41: M=Ni²⁺, R₁=Et, R₂═R₃═R₄═R₅═H;
42: M=Ni²⁺, R₁=Et, R₅=Me, R₂═R₃═R₄═H;
43: M=Ni²⁺, R₁═R₅=Et, R₂═R₃═R₄═H;
44: M=Ni²⁺, R₁=iPr, R₂═R₃═R₄═R₅═H;
45: M=Ni²⁺, R₁═R₅=iPr, R₂═R₃═R₄═H.

In one further preferred embodiment of said catalyst composition, R₁ and R₅ in the main catalyst are ethyl, and R₂-R₄ in the main catalyst are hydrogens.

The preparation of the main catalyst of the present invention is already known, e.g. see CN1850339A; the preparation process disclosed therein is incorporated herein by reference.

The process for preparing the main catalyst as shown in Formula (I) according to the present invention comprises the following steps:
1) Enabling 2-acetyl-1,10-phenanthroline reacted with substituted aniline, wherein the substituent is selected from (C₁-C₆) alkyl, halogen, (C₁-C₆) alkoxyl or nitro group, and then obtaining 2-imino-1,10-phenanthrolinyl ligand; and
2) Enabling said 2-imino-1,10-phenanthrolinyl ligand obtained in step 1) reacted with FeCl₂.4H₂O, CoCl₂ or NiCl₂.6H₂O respectively, thus obtaining the corresponding complex.

In particular, the main catalyst according to the present invention is prepared as follows:
1. General approach to synthesize ligand
   1) Refluxing the reaction mixture of 2-acetyl-1,10-phenanthroline and (C₁-C₆) alkyl substituted aniline in ethanol with p-toluene sulfonic acid as catalyst for 1 to 2 days; after concentration, the reaction solution is passed through a basic alumina column, eluted with petroleum ether/ethyl acetate (4:1); the second fraction is the desired product; removing the solvent and then obtaining a yellow solid of 2-imino-1,10-phenanthrolinyl ligand;
   2) Refluxing a reaction mixture of 2-acetyl-1,10-phenanthroline and F, (C₁-C₆) alkoxyl or nitro substituted aniline in toluene with p-toluene sulfonic acid as catalyst and molecular sieve or anhydrous sodium sulfate as dehydrant for 1 day; after filtration and toluene removal, the reaction mixture is passed through a basic alumina column, eluted with petroleum ether/ethyl acetate (4:1); the second fraction is the desired product; removing the solvent and then obtaining a yellow solid of 2-imino-1,10-phenanthrolinyl ligand;
   3) Heating 2-acetyl-1,10-phenanthroline and Cl or Br substituted aniline at the temperature of 140 to 150° C. with p-toluene sulfonic acid as catalyst and ethyl orthosilicate as solvent and dehydrant for 1 day; after removal of ethyl orthosilicate under a reduced pressure, the reaction mixture is passed through a basic alumina column, and eluted with petroleum ether/ethyl acetate (4:1); the second fraction is the desired product; removing the solvent and then obtaining a yellow solid of 2-imino-1,10-phenanthrolinyl ligand.
   Said alkyl substituted aniline is preferably 2,6-diethyl aniline.
   All of the above synthesized 2-imino-1,10-phenanthrolinyl ligands have been confirmed by NMR, IR and elemental analysis.
2. General approach to synthesize iron (II), cobalt (II) or nickel (II) complexes
   The solution of FeCl₂.4H₂O, CoCl₂ or NiCl₂.6H₂O in ethanol is added dropwise to the solution of 2-imino-1,10-phenanthrolinyl ligand at a molar ratio of 1:1 to 1:1.2. The reaction mixture is stirred at room temperature, and the precipitate is filtered, washed with ether and then dried, thus obtaining the 2-imino-1,10-phenanthrolinyl complex. The complexes 1 to 45 are confirmed by IR spectrum characterization and elemental analysis.

According to another aspect of the present invention, a process for ethylene oligomerization is provided, wherein a catalyst composition comprising 2-imino-1,10-phenanthroline coordinated iron (II), cobalt (II) or nickel (II) chloride as shown in Formula (I) as the main catalyst and triethylaluminum as the cocatalyst is used, and the molar ratio of aluminum in the cocatalyst to the central metal in the main catalyst ranges from 30 to less than 200:

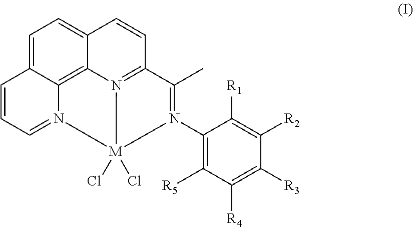

wherein M is the central metal, selected from Fe²⁺, Co²⁺ and Ni²⁺; R₁-R₅ are independently selected from hydrogen, (C₁-C₆) alkyl, halogen, (C₁-C₆) alkoxyl and nitro group.

In an advantageous embodiment of said process for ethylene oligomerization, the molar ratio of aluminum in the cocatalyst to the central metal (i.e. Fe²⁺, Co²⁺ or Ni²⁺) in the main catalyst ranges from 50 to less than 200, preferably from 100 to 199.8, more preferably from 148 to 196, and most preferably from 178 to 196.

In a preferred embodiment of said process for ethylene oligomerization, R₁-R₅ in the main catalyst are independently selected from hydrogen, methyl, ethyl, isopropyl, fluoro, chloro, bromo, methoxyl, ethoxyl and nitro group.

In a further preferred embodiment of said process for ethylene oligomerization, R₁ and R₅ in the main catalyst are ethyl, and R₂-R₄ in the main catalyst are hydrogens.

In another advantageous embodiment of said process for ethylene oligomerization, M and R₁-R₅ in the main catalyst are defined as follows:
1: M=Fe²⁺, R₁=Me, R₂═R₃═R₄═R₅═H;
2: M=Fe²⁺, R₂=Me, R₁═R₃═R₄═R₅═H;
3: M=Fe²⁺, R₃=Me, R₁═R₂═R₄═R₅═H;
4: M=Fe²⁺, R₁═R₂=Me, R₃═R₄═R₅═H;
5: M=Fe²⁺, R₁═R₃=Me, R₂═R₄═R₅═H;
6: M=Fe²⁺, R₁═R₄=Me, R₂═R₃═R₅═H;
7: M=Fe²⁺, R₁═R₅=Me, R₂═R₃═R₄═H;
8: M=Fe²⁺, R₂═R₃=Me, R₁═R₄═R₅═H;
9: M=Fe²⁺, R₂═R₄=Me, R₁═R₃═R₅═H;
10: M=Fe²⁺, R₁═R₃═R₅=Me, R₂═R₄═H;
11: M=Fe²⁺, R₁=Et, R₂═R₃═R₄═R₅═H;
12: M=Fe²⁺, R₁=Et, R₅=Me, R₂═R₃═R₄═H;
13: M=Fe²⁺, R₁═R₅=Et, R₂═R₃═R₄═H;
14: M=Fe²⁺, R₁=iPr, R₂═R₃═R₄═R₅═H;
15: M=Fe²⁺, R₁═R₅=iPr, R₂═R₃═R₄═H;
16: M=Co²⁺, R₁=Me, R₂═R₃═R₄═R₅═H;
17: M=Co²⁺, R₂=Me, R₁═R₃═R₄═R₅═H;
18: M=Co²⁺, R₃=Me, R₁═R₂═R₄═R₅═H;
19: M=Co²⁺, R₁═R₂=Me, R₃═R₄═R₅═H;
20: M=Co²⁺, R₁═R₃=Me, R₂═R₄═R₅═H;
21: M=Co²⁺, R₁═R₄=Me, R₂═R₃═R₅═H;
22: M=Co²⁺, R₁═R₅=Me, R₂═R₃═R₄═H;
23: M=Co²⁺, R₂═R₃=Me, R₁═R₄═R₅═H;
24: M=Co²⁺, R₂═R₄=Me, R₁═R₃═R₅═H;
25: M=Co²⁺, R₁═R₃═R₅=Me, R₂═R₄═H;
26: M=Co²⁺, R₁=Et, R₂═R₃═R₄═R₅═H;
27: M=Co²⁺, R₁=Et, R₅=Me, R₂═R₃═R₄═H;
28: M=Co²⁺, R₁═R₅=Et, R₂═R₃═R₄═H;
29: M=Co²⁺, R₁=iPr, R₂═R₃═R₄═R₅═H;
30: M=Co²⁺, R₁═R₅=iPr, R₂═R₃═R₄═H;
31: M=Ni²⁺, R₁=Me, R₂═R₃═R₄═R₅═H;
32: M=Ni²⁺, R₂=Me, R₁═R₃═R₄═R₅═H;
33: M=Ni²⁺, R₃=Me, R₁═R₂═R₄═R₅═H;
34: M=Ni²⁺, R₁═R₂=Me, R₃═R₄═R₅═H;
35: M=Ni²⁺, R₁═R₃=Me, R₂═R₄═R₅═H;
36: M=Ni²⁺, R₁═R₄=Me, R₂═R₃═R₅═H;
37: M=Ni²⁺, R₁═R₅=Me, R₂═R₃═R₄═H;
38: M=Ni²⁺, R₂═R₃=Me, R₁═R₄═R₅═H;
39: M=Ni²⁺, R₂═R₄=Me, R₁═R₃═R₅═H;
40: M=Ni²⁺, R₁═R₃═R₅=Me, R₂═R₄═H;
41: M=Ni²⁺, R₁=Et, R₂═R₃═R₄═R₅═H;
42: M=Ni²⁺, R₁=Et, R₅=Me, R₂═R₃═R₄═H;
43: M=Ni²⁺, R₁═R₅=Et, R₂═R₃═R₄═H;
44: M=Ni²⁺, R₁=iPr, R₂═R₃═R₄═R₅═H;
45: M=Ni²⁺, R₁═R₅=iPr, R₂═R₃═R₄═H.

The reaction condition of said oligomerization process is known to one skilled in the art. A preferred example for the process is as follows: adding said catalyst composition and organic solvent into a reactor; carrying out the oligomerization reaction with an ethylene pressure of 0.1 to 30 MPa and a reaction temperature of 20 to 150° C. for 30 to 100 min; then cooling to −10 to 10° C., and collecting a small amount of reaction mixture and neutralizing it with 5% aqueous hydrogen chloride for gas chromatography (GC) analysis.

In the above oligomerization process, the reaction temperature is preferably from 20 to 80° C., the reaction pressure is preferably from 1 to 5 MPa, and the reaction time is advantageously from 30 to 60 min.

In the above oligomerization process, the organic solvent is selected from toluene, cyclohexane, ether, tetrahydrofuran, ethanol, benzene, xylene, dicholomethane and so on, and preferably toluene.

Using the above oligomerization process to oligomerize ethylene, the obtained oligomerization products include C₄ olefine, C₆ olefines, C₈ olefines, C₁₀ olefines, C₁₂ olefines, C₁₄ olefines, C₁₆ olefines, C₁₈ olefines, C₂₀ olefines, C₂₂ olefines and so on, and the selectivity of alpha olefins is in excess of 95%. After the ethylene oligomerization, a small amount of the reaction mixture is collected and neutralized with 5% aqueous hydrogen chloride for GC analysis. Result shows the oligomerization activity is in excess of 10⁶ g·mol⁻¹·h⁻¹, and the distribution of the products is more reasonable. Moreover, the residual reaction mixture is neutralized with a solution of 5% aqueous hydrochloric acid in ethanol, and no polymer formation is observed.

In the above oligomerization process, in which a catalyst composition comprising low cost triethylaluminum (the price of which is just a fraction of that of methylaluminoxane) as the cocatalyst and 2-imino-1,10-phenanthroline coordinated iron (II), cobalt (II) or nickel (II) chloride as the main catalyst is used, at the molar ratio of aluminum in the cocatalyst to central metal in the main catalyst ranging from 30 to less than 200, the catalytic activity is acceptable even with a low amount of cocatalyst, thus having a high practicability.

According to the present invention, another process for ethylene oligomerization is provided, wherein a catalyst composition comprising 2-imino-1,10-phenanthroline coordinated iron (II), cobalt (II) or nickel (II) chloride as shown in Formula (I) as main catalyst and triethylaluminum as cocatalyst is used, and the reaction temperature of ethylene oligomerization is from −10 to 19° C.:

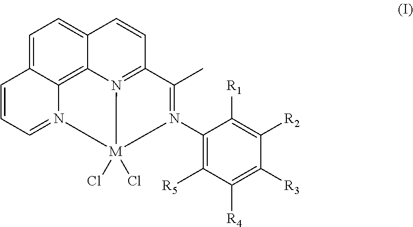

wherein M is the central metal, preferably selected from Fe²⁺, Co²⁺ and Ni²⁺; R₁-R₅ are independently selected from hydrogen, (C₁-C₆) alkyl, halogen, (C₁-C₆) alkoxyl and nitro group.

In a preferred embodiment of said process for ethylene oligomerization, R₁-R₅ in the main catalyst are independently selected from hydrogen, methyl, ethyl, isopropyl, fluoro, chloro, bromo, methoxyl, ethoxyl and nitro group.

In a further preferred embodiment of said process for ethylene oligomerization, R₁ and R₅ in the main catalyst are ethyl group, and R₂-R₄ in the main catalyst are hydrogen atoms.

In an advantageous embodiment of said process for oligomerization of ethyl, M and R₁-R₅ in the main catalyst are defined as follows:
1: M=Fe²⁺, R₁=Me, R₂═R₃═R₄═R₅═H;
2: M=Fe²⁺, R₂=Me, R₁═R₃═R₄═R₅═H;
3: M=Fe²⁺, R₃=Me, R₁═R₂═R₄═R₅═H;
4: M=Fe²⁺, R₁═R₂=Me, R₃═R₄═R₅═H;
5: M=Fe²⁺, R₁═R₃=Me, R₂═R₄═R₅═H;
6: M=Fe²⁺, R₁═R₄=Me, R₂═R₃═R₅═H;
7: M=Fe²⁺, R₁═R₅=Me, R₂═R₃═R₄═H;
8: M=Fe²⁺, R₂═R₃=Me, R₁═R₄═R₅═H;
9: M=Fe²⁺, R₂═R₄=Me, R₁═R₃═R₅═H;
10: M=Fe²⁺, R₁═R₃═R₅=Me, R₂═R₄═H;
11: M=Fe²⁺, R₁=Et, R₂═R₃═R₄═R₅═H;
12: M=Fe²⁺, R₁=Et, R₅=Me, R₂═R₃═R₄═H;
13: M=Fe²⁺, R₁═R₅=Et, R₂═R₃═R₄═H;
14: M=Fe²⁺, R₁=iPr, R₂═R₃═R₄═R₅═H;
15: M=Fe²⁺, R₁═R₅=iPr, R₂═R₃═R₄═H;
16: M=Co²⁺, R₁=Me, R₂═R₃═R₄═R₅═H;
17: M=Co²⁺, R₂=Me, R₁═R₃═R₄═R₅═H;
18: M=Co²⁺, R₃=Me, R₁═R₂═R₄═R₅═H;
19: M=Co²⁺, R₁═R₂=Me, R₃═R₄═R₅═H;
20: M=Co²⁺, R₁═R₃=Me, R₂═R₄═R₅═H;
21: M=Co²⁺, R₁═R₄=Me, R₂═R₃═R₅═H;
22: M=Co²⁺, R₁═R₅=Me, R₂═R₃═R₄═H;
23: M=Co²⁺, R₂═R₃=Me, R₁═R₄═R₅═H;
24: M=Co²⁺, R₂═R₄=Me, R₁═R₃═R₅═H;
25: M=Co²⁺, R₁═R₃═R₅=Me, R₂═R₄═H;
26: M=Co²⁺, R₁=Et, R₂═R₃═R₄═R₅═H;
27: M=Co²⁺, R₁=Et, R₅=Me, R₂═R₃═R₄═H;
28: M=Co²⁺, R₁═R₅=Et, R₂═R₃═R₄═H;
29: M=Co²⁺, R₁=iPr, R₂═R₃═R₄═R₅═H;
30: M=Co²⁺, R₁═R₅=iPr, R₂═R₃═R₄═H;
31: M=Ni²⁺, R₁=Me, R₂═R₃═R₄═R₅═H;
32: M=Ni²⁺, R₂=Me, R₁═R₃═R₄═R₅═H;
33: M=Ni²⁺, R₃=Me, R₁═R₂═R₄═R₅═H;
34: M=Ni²⁺, R₁═R₂=Me, R₃═R₄═R₅═H;
35: M=Ni²⁺, R₁═R₃=Me, R₂═R₄═R₅═H;
36: M=Ni²⁺, R₁═R₄=Me, R₂═R₃═R₅═H;
37: M=Ni²⁺, R₁═R₅=Me, R₂═R₃═R₄═H;
38: M=Ni²⁺, R₂═R₃=Me, R₁═R₄═R₅═H;
39: M=Ni²⁺, R₂═R₄=Me, R₁═R₃═R₅═H;
40: M=Ni²⁺, R₁═R₃═R₅=Me, R₂═R₄═H;
41: M=Ni²⁺, R₁=Et, R₂═R₃═R₄═R₅═H;
42: M=Ni²⁺, R₁=Et, R₅=Me, R₂═R₃═R₄═H;
43: M=Ni²⁺, R₁═R₅=Et, R₂═R₃═R₄═H;
44: M=Ni²⁺, R₁=iPr, R₂═R₃═R₄═R₅═H;
45: M=Ni²⁺, R₁═R₅=iPr, R₂═R₃═R₄═H.

The above oligomerization process can be carried out preferably as follows: adding organic solvent and said catalyst composition into a reactor; carrying out the oligomerization reaction with an ethylene pressure of 0.1 to 30 MPa and a reaction temperature of −10 to 19° C. for 30 to 100 min; then at a temperature of −10 to 10° C. collecting a small amount of the reaction mixture and neutralizing it with 5% aqueous hydrogen chloride for gas chromatography (GC) analysis.

In the above oligomerization process, the main catalyst is usually used in the form of solution. Suitable solvents can be conventional solvent, e.g. selected from toluene, cyclohexane, ether, tetrahydrofuran, ethanol, benzene, xylene and dicholomethane, preferably toluene.

In the above oligomerization process, the reaction temperature is preferably from −10 to 15° C., more preferably 0 to 15° C., and most preferably 5 to 10° C. The reaction time is advantageously from 30 to 60 min, and the reaction pressure is advantageously from 1 to 5 MPa.

In the above oligomerization process, the molar ratio of aluminum in the cocatalyst to the central metal in the main catalyst ranges from 49 to 500, preferably from 100 to 400, more preferably from 200 to 300, and most preferably 300.

In the above oligomerization process, the organic solvent is selected from toluene, cyclohexane, ether, tetrahydrofuran, ethanol, benzene, xylene and dicholomethane, preferred toluene.

Using the described process to oligomerize ethylene, the obtained oligomerization products include C₄ olefin, C₆ olefins, C₈ olefins, C₁₀ olefins, C₁₂ olefins, C₁₄ olefins, C₁₆ olefins, C₁₈ olefins, C₂₀ olefins, C₂₂ olefins and so on, with a high alpha olefin selectivity being in excess of 96% and a high oligomerization activity. Moreover, the residual reaction mixture is neutralized with a solution of 5% aqueous hydrochloric acid in ethanol, and thus only a few polymers are generated.

In the above process for ethylene oligomerization, with a catalyst composition comprising 2-imino-1,10-phenanthroline coordinated iron (II), cobalt (II) or nickel (II) chloride as main catalyst and low cost triethylaluminum as cocatalyst being used, it is surprisingly found that the ethylene catalytic activity is still high even with a low amount of cocatalyst at a low temperature of −10 to 19° C. As such, the present invention provides a new approach for ethylene oligomerization.

Compared with the prior arts, the catalyst composition according to the present invention comprising 2-imino-1,10-phenanthroline coordinated iron (II), cobalt (II) or nickel (II) chloride as the main catalyst and triethylaluminum (AlEt₃), the price of which is just a fraction of that of methylaluminoxane, as cocatalyst is used in the process for ethylene oligomerization, with the results that the catalytic activity is acceptable with high selectivity of alpha olefins, and the amount of cocatalyst is low, so that the catalyst effect is cost-effective. Therefore, the catalyst composition of the present invention is quite industrially applicable. According to the present invention, the technical bias that triethylaluminum is improper as cocatalyst for ethylene oligomerization is overcome, the reaction condition is optimized, and the cost of ethylene oligomerization is significantly reduced. In view of the catalysis effect and the cost, the present invention is highly applicable in industry.

### EXAMPLES

The following examples are provided merely as preferred examples of the present invention, with no restriction to the scope of the present invention in any way. All the changes and modifications made based on the present invention are within the scope of the present invention.

### Example 1

### 1. Main Catalyst Preparation

A reaction solution of 0.4445 g (2 mmol) 2-acetyl-1,10-phenanthroline and 0.4175 g (2.8 mmol) 2,6-diethyl aniline is refluxed in 30 ml ethanol for 1 day, in which 40 mg p-toluene sulphonic acid is added as catalyst and 2 g molecular sieve of 4 Å is added as dehydration agent. After filtration, the solvent is removed; the residue is dissolved in dichloromethane, then passed through a basic alumina column, and eluted with petroleum ether/ethyl acetate (4:1). The second fraction is the desired product, and after removal of solvent, a yellow solid of 0.6 g 2-acetyl-1,10-phenanthrolinyl (2,6-diethylanil) ligand is obtained with a yield of 84%. The analysis of Nuclear Magnetic Resonance Spectroscopy: ¹H-NMR (300 Hz, CDCl₃), δ 9.25 (dd, J=3.0 Hz, 1H); 8.80 (d, J=8.3 Hz, 1H); 8.35 (d, J=8.3 Hz, 1H); 8.27 (dd, J=7.8 Hz, 1H); 7.86 (s, 2H); 7.66 (s, 2H); 7.15 (d, J=7.6 Hz, 2H); 6.96 (t, J=7.5 Hz, 1H); 2.58 (s, 3H, CH₃); 2.43 (m, 4H, CH₂CH₃); 1.16 (t, J=7.5 Hz, 6H, CH₂CH₃). Anal. Calc. for C₂₄H₂₃N₃ (353.46): C, 81.55; H, 6.56; N, 11.89. Found: C, 80.88; H, 6.59; N, 11.78.

A 5 ml solution of 48 mg FeCl₂.4H₂O (0.24 mmol) in anhydrous ethanol is added dropwise into a 5 ml solution of 70.6 mg 2-acetyl-1,10-phenanthrolinyl (2,6-diethylanil) ligand (0.2 mmol) in anhydrous ethanol. After stirring for 6 h at room temperature, the resulting precipitate is filtered, washed with ether and dried, thus obtaining a dark green powder solid of 2-acetyl-1,10-phenanthroline (2,6-diethylanil)FeCl₂ complex at a yield of 95%. Anal. Calc. for C₂₄H₂₃Cl₂FeN₃ (480.21): C, 60.03; H, 4.83; N, 8.75. Found: C, 59.95; H, 4.92; N, 8.80.

### 2. Ethylene Oligomerization Reaction

Toluene, a 0.53 ml solution (0.74 mol/l) of triethylaluminum in toluene and a 8 ml solution of the main catalyst, i.e., 2-acetyl-1,10-phenanthroline(2,6-diethylanil)FeCl₂ (2.0 μmol), in toluene are added into a 300 ml stainless steel autoclave, the total volume being 100 ml and Al/Fe=196. Ethylene is added into the autoclave when the temperature reaches 40° C., the ethylene pressure being kept at 1 MPa and the reaction being carried out for 30 min under stirring. A small amount of reaction mixture is collected by syringe and neutralized by 5% aqueous hydrogen chloride. The neutralized solution is then analyzed by gas chromatography (GC) analysis. Result shows that the oligomerization activity is 2.02×10⁶ g·mol⁻¹(Fe)·h⁻¹, and the contents of oligomers are as follows: C₄, 12.0%; C₆-C₁₀, 64.7%; C₆-C₁₈, 87.0% (the content of linear alpha olefins is 98.0%); C₂₀-C₂₈, 1.0%. The residual reaction mixture is neutralized by a solution of 5% aqueous hydrochloric acid in ethanol, no polymer formation being observed. The result is shown in Table 1.

### Example 2

The ethylene oligomerization reaction is carried out using the complex prepared in Example 1 as the main catalyst and triethylaluminum as the cocatalyst. Example 2 differs from Example 1 in that, the amount of the solution of triethylaluminum in toluene is 0.54 ml (0.74 mol/l) and Al/Fe=199.8. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 2.02×10⁶ g·mol⁻¹(Fe)·h⁻¹, and the contents of oligomers are as follows: C₄, 12.1%; C₆-C₁₀, 64.5%; C₆-C₁₈, 86.8% (the content of linear alpha olefins is 97.5%); C₂₀-C₂₈, 1.1%. The residual reaction mixture is neutralized by a solution of 5% aqueous hydrochloric acid in ethanol, no polymer formation being observed. The result is shown in Table 1.

### Example 3

The ethylene oligomerization reaction is carried out using the complex prepared in Example 1 as the main catalyst and triethylaluminum as the cocatalyst. Example 3 differs from Example 1 in that, the amount of the solution of triethylaluminum in toluene is 0.51 ml (0.74 mol/l) and Al/Fe=189. With the ethylene pressure being kept at 1 MPa, the reaction is carried out at 40° C. for 30 min under stirring. A small amount of reaction mixture is collected by syringe and neutralized by 5% aqueous hydrogen chloride, the neutralized solution is then analyzed by GC analysis. Result shows that, the oligomerization activity is 1.98×10⁶ g·mol⁻¹(Fe)·h⁻¹, and the contents of oligomers are as follows: C₄, 11.6%; C₆-C₁₀, 64.8%; C₆-C₁₈, 86.9% (in which the content of linear alpha olefins is 98.0%); C₂₀-C₂₈, 1.5%. The residual reaction mixture is neutralized by a solution of 5% aqueous hydrochloric acid in ethanol, no polymer formation being observed. The result is shown in Table 1.

### Example 4

The ethylene oligomerization reaction is carried out using the complex prepared in Example 1 as the main catalyst and triethylaluminum as the cocatalyst. Example 4 differs from Example 1 in that, the amount of the solution of triethylaluminum in toluene is 0.48 ml (0.74 mol/l) and Al/Fe=178. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 1.98×10⁶ g·mol⁻¹(Fe)·h⁻¹, and the contents of oligomers are as follows: C₄, 10.5%; C₆-C₁₀, 65.1%; C₆-C₁₈, 87.7% (in which the content of linear alpha olefins is 98.3%); C₂₀-C₂₈, 1.8%. The residual reaction mixture is neutralized by a solution of 5% aqueous hydrochloric acid in ethanol, no polymer formation being observed. The result is shown in Table 1.

### Example 5

The ethylene oligomerization reaction is carried out using the complex prepared in Example 1 as the main catalyst and triethylaluminum as the cocatalyst. Example 5 differs from Example 1 in that, the amount of the solution of triethylaluminum in toluene is 0.4 ml (0.74 mol/l) and Al/Fe=148. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 1.21×10⁶ g·mol⁻¹(Fe)·h⁻¹, and the contents of oligomers are as follows: C₄, 24.7%; C₆-C₁₀, 57.4%; C₆-C₁₈, 72.7% (in which the content of linear alpha olefins is 92.9%); C₂₀-C₂₈, 2.6%. The residual reaction mixture is neutralized by a solution of 5% aqueous hydrochloric acid in ethanol, no polymer formation being observed. The result is shown in Table 1.

### Example 6

The ethylene oligomerization reaction is carried out using the complex prepared in Example 1 as the main catalyst and triethylaluminum as the cocatalyst. Example 6 differs from Example 1 in that, the amount of the solution of triethylaluminum in toluene is 0.81 ml (0.25 mol/l) and Al/Fe=101. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 1.01×10⁶ g·mol⁻¹(Fe)·h⁻¹, and the contents of oligomers are as follows: C₄, 21.6%; C₆-C₁₀, 53.6%; C₆-C₁₈, 75.3% (in which the content of linear alpha olefins is 89.9%); C₂₀-C₂₈, 3.1%. The residual reaction mixture is neutralized by a solution of 5% aqueous hydrochloric acid in ethanol, no polymer formation being observed. The result is shown in Table 1.

### Example 7

The ethylene oligomerization is carried out using the complex prepared in Example 1 as the main catalyst and triethylaluminum as the cocatalyst. Example 7 differs from Example 1 lie in that, the amount of the solution of triethylaluminum in toluene is 0.4 ml (0.25 mol/l) and Al/Fe=50. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 0.12×10⁶ g·mol⁻¹(Fe)·h⁻¹, and the contents of oligomers are as follows: C₄, 7.4%; C₆-C₁₀, 86.8%; C₆-C₁₈, 92.6% (in which the content of linear alpha olefins is 92.5%); C₂₀-C₂₈, 0%. The residual reaction mixture is neutralized by a solution of 5% aqueous hydrochloric acid in ethanol, no polymer formation being observed. The result is shown in Table 1.

### Example 8

The ethylene oligomerization reaction is carried out using the complex prepared in Example 1 as the main catalyst and triethylaluminum as the cocatalyst. Example 8 differs from Example 1 is that, the amount of the solution of triethylaluminum in toluene is 0.24 ml (0.25 mol/l) and Al/Fe=30. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 0.08×10⁶ g·mol⁻¹(Fe)·h⁻¹, and the contents of oligomers are as follows: C₄, 6.9%; C₆-C₁₀, 87.1%; C₆-C₁₈, 93.1% (in which the content of linear alpha olefins is 91.5%); C₂₀-C₂₈, 0%. The residual reaction mixture is neutralized by a solution of 5% aqueous hydrochloric acid in ethanol, no polymer formation being observed. The result is shown in Table 1.

### Example 9

In Example 9 the preparation process of the main catalyst as in Example 1 is used. Example 9 differs from Example 1 in that, a 5 ml solution of 31.2 mg CoCl₂ (0.24 mmol) in anhydrous ethanol is added dropwise into a 5 ml solution of 70.6 mg 2-acetyl-1,10-phenanthrolinyl (2,6-diethylanil) ligand (0.2 mmol) in anhydrous ethanol. After stirring for 6 h at room temperature, the resulting precipitate is filtered, washed with ether and dried, thus obtaining a brown solid of 2-acetyl-1,10-phenanthroline (2,6-diethylanil)CoCl₂ complex at a yield of 95%. Anal. Calc. for C₂₄H₂₃Cl₂CoN₃ (483.29): C, 59.64; H, 4.80; N, 8.69. Found: C, 59.69; H, 4.86; N, 8.62.

The process for ethylene oligomerization is repeated as in Example 1, and the cocatalyst is still triethylaluminum. Toluene, a 0.53 ml solution (0.74 mol/l) of triethylaluminum in toluene and a 8 ml solution of 2-acetyl-1,10-phenanthroline (2,6-diethylanil)CoCl₂ (2.0 μmol) in toluene are added into a 300 ml stainless steel autoclave, the total volume being 100 ml and Al/Co=196. Ethylene is added into the autoclave when the temperature reaches 40° C., the ethylene pressure being kept at 1 MPa and the reaction being carried out for 30 min under stirring. A small amount of reaction mixture is collected by syringe and neutralized by 5% aqueous hydrogen chloride. The neutralized solution is then analyzed by GC analysis. Result shows that, the oligomerization activity is 1.51×10⁶ g·mol⁻¹(Co)·h⁻¹, and the content of oligomers is: C₄, 100%. The residual reaction mixture is neutralized by a solution of 5% aqueous hydrochloric acid in ethanol, no polymer formation being observed. The result is shown in Table 1.

### Example 10

In Example 10 the preparation process of the main catalyst as in Example 1 is used. Example 10 differs from Example 1 in that, a 5 ml solution of 57.0 mg NiCl₂.6H₂O (0.24 mmol) in anhydrous ethanol is added dropwise into a 5 ml solution of 70.6 mg 2-acetyl-1,10-phenanthrolinyl (2,6-diethylanil) ligand (0.2 mmol) in anhydrous ethanol. After stirring for 6 h at room temperature, the resulting precipitate is filtered, washed with ether and dried, thus obtaining a yellow-brown solid of 2-acetyl-1,10-phenanthroline (2,6-diethylanil)NiCl₂ complex at a yield of 96%. Anal. Calc. for C₂₄H₂₃Cl₂NiN₃ (483.05): C, 59.67; H, 4.80; N, 8.70. Found: C, 59.64; H, 4.82; N, 8.53.

The process for ethylene oligomerization is repeated as in Example 1, and the cocatalyst is still triethylaluminum. Toluene, a 0.53 ml solution (0.74 mol/l) of triethylaluminum in toluene and a 8 ml solution of 2-acetyl-1,10-phenanthroline (2,6-diethylanil)NiCl₂ (2.0 μmol) in toluene are added into a 300 ml stainless steel autoclave, the total volume being 100 ml and Al/Ni=196. Ethylene is added into the autoclave at 40° C., the ethylene pressure being kept at 1 MPa and the reaction being carried out for 30 min under stirring. A small amount of reaction mixture is collected by syringe and neutralized by 5% aqueous hydrogen chloride. The neutralized solution is then analyzed by GC analysis. Result shows the oligomerization activity is 1.40×10⁶ g·mol⁻¹(Ni)·h⁻¹, and the content of oligomers is: C₄, 100%. The residual reaction mixture is neutralized by a solution of 5% aqueous hydrochloric acid in ethanol, no polymer formation being observed. The result is shown in Table 1.

### Example 11

The Ethylene oligomerization reaction is carried out using the complex prepared in Example 1 as the main catalyst and triethylaluminum as the cocatalyst. The amount of the solution (0.74 mol/l) of triethylaluminum in toluene is 0.53 ml, and Al/Fe=196. Example 11 differs from Example 1 in that, with the ethylene pressure being kept at 2 MPa the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 3.21×10⁶ g·mol⁻¹(Fe)·h⁻¹, and the contents of oligomers are as follows: C₄, 19.40%; C₆-C₁₀, 53.02%; C₆-C₁₈, 75.68% (in which the content of linear alpha olefins is 96.9%); C₂₀-C₂₈, 4.92%. The residual reaction mixture is neutralized by a solution of 5% aqueous hydrochloric acid in ethanol, no polymer formation being observed. The result is shown in Table 1.

### Example 12

The ethylene oligomerization reaction is carried out using the complex prepared in Example 1 as the main catalyst and triethylaluminum as the cocatalyst. Example 12 differs from Example 1 in that, the amount of the solution of triethylaluminum in toluene is 0.54 ml (0.74 mol/l) and Al/Fe=199.8; with the ethylene pressure being kept at 2 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 3.83×10⁶ g·mol⁻¹(Fe)·h⁻¹, and the contents of oligomers are as follows: C₄, 21.05%; C₆-C₁₀, 52.37%; C₆-C₁₈, 73.36% (in which the content of linear alpha olefins is 97.5%); C₂₀-C₂₈, 5.59%. The residual reaction mixture is neutralized by a solution of 5% aqueous hydrochloric acid in ethanol, no polymer formation being observed. The result is shown in Table 1.

### Example 13

The ethylene oligomerization reaction is carried out using the complex prepared in Example 1 as the main catalyst and triethylaluminum as the cocatalyst, in which the amount of the solution of triethylaluminum in toluene is 0.53 ml (0.74 mol/l) and Al/Fe=196. Example 13 differs from Example 1 in that, with the ethylene pressure being kept at 3 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 6.40×10⁶ g·mol⁻¹(Fe)·h⁻¹, and the contents of oligomers are as follows: C₄, 17.5%; C₆-C₁₀, 46.2%; C₆-C₁₈, 71.5% (in which the content of linear alpha olefins is 98.7%); C₂₀-C₂₈, 11.0%. The residual reaction mixture is neutralized by a solution of 5% aqueous hydrochloric acid in ethanol, no polymer formation being observed. The result is shown in Table 1.

### Example 14

The ethylene oligomerization reaction is carried out using the complex prepared in Example 1 as the main catalyst and triethylaluminum as the cocatalyst. Example 14 differs from Example 1 in that, the amount of the solution of triethylaluminum in toluene is 0.4 ml (0.74 mol/l) and Al/Fe=148; and with the ethylene pressure being kept at 3 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 5.21×10⁶ g·mol⁻¹(Fe)·h⁻¹, and the contents of oligomers are as follows: C₄, 19.5%; C₆-C₁₀, 53.4%; C₆-C₁₈, 75.8% (in which the content of linear alpha olefins is 98.4%); C₂₀-C₂₈, 4.7%. The residual reaction mixture is neutralized by a solution of 5% aqueous hydrochloric acid in ethanol, no polymer formation being observed. The result is shown in Table 1.

### Comparative Example 1

The ethylene oligomerization reaction is carried out using the complex prepared in Example 1 as the main catalyst and triethylaluminum as the cocatalyst. Comparative Example 1 differs from Example 1 in that, the amount of the solution of triethylaluminum in toluene is 1.35 ml (0.74 mol/l) and Al/Fe=500. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 0.88×10⁶ g·mol⁻¹(Fe)·h⁻¹, and the contents of oligomers are as follows: C₄, 37.0%; C₆-C₁₀, 52.0%; C₆-C₁₈, 63.0% (in which the content of linear alpha olefins is 91.5%); C₂₀-C₂₈, 0%. The residual reaction mixture is neutralized by a solution of 5% aqueous hydrochloric acid in ethanol, no polymer formation being observed. The result is shown in Table 1.

### Comparative Example 2

The example 34 disclosed in CN1850339A is incorporated herein by reference. In this Comparative Example 2, 2-acetyl-1,10-phenanthroline (2,6-diethylanil)FeCl₂ is used as main catalyst and triethylaluminum is used as cocatalyst. The process for ethylene oligomerization is as follows: 1000 ml toluene, a 5.0 ml solution (1.0 mol/l) of triethylaluminum in hexane and a 10 ml solution of 2-imino-1,10-phenanthroline (2,6-diethylanil) coordinated iron (II) chloride (10 μmol) in toluene are added into a 2000 ml stainless steel autoclave. Under a mechanical stirring of 350 rev/min, ethylene is added into the autoclave at 40° C., and the oligomerization reaction begins. With the ethylene pressure being kept at 1 MPa, the reaction is carried out at 40° C. for 60 min under stirring. A small amount of reaction mixture is collected by syringe and neutralized by 5% aqueous hydrogen chloride. The neutralized solution is then analyzed by GC analysis. Result shows that, the oligomerization activity is 0.271×10⁶ g·mol⁻¹(Fe)·h⁻¹, and the contents of oligomers are as follows: C₄, 39.3%; C₆, 29.3%; C₈-C₂₂, 31.4%. The residual reaction mixture is neutralized by a solution of 5% aqueous hydrochloric acid in ethanol, no polymer formation being observed. The result is shown in Table 1.

### Comparative Example 3

The ethylene oligomerization reaction is carried out using the complex prepared in Example 1 as the main catalyst and triethylaluminum as the cocatalyst. Comparative Example 3 differs from Example 1 in that, the amount of the solution of triethylaluminum in toluene is 2.70 ml (0.74 mol/l) and Al/Fe=1000. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 0.18×10⁶ g·mol⁻¹(Fe)·h⁻¹, and the contents of oligomers are as follows: C₄, 43.9%; C₆-C₁₀, 50.9%; C₆-C₁₈, 55.5% (in which the content of linear alpha olefins is 84.3%); C₂₀-C₂₈, 0.6%. The residual reaction mixture is neutralized by a solution of 5% aqueous hydrochloric acid in ethanol, no polymer formation being observed. The result is shown in Table 1.

### Comparative Example 4

The ethylene oligomerization reaction is carried out using the complex prepared in Example 1 as the main catalyst and the process in Example 1. Comparative Example 4 differs from Example 1 in that, methylaluminoxane is used as the cocatalyst, the amount of the solution of methylaluminoxane in toluene is 0.26 ml (1.5 mol/l) and Al/Fe=195. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 2.5×10⁶ g·mol⁻¹(Fe)·h⁻¹, and the contents of oligomers are as follows: C₄, 14.2%; C₆-C₁₀, 44.9%; C₆-C₁₈, 74.1% (in which the content of linear alpha olefins is 89.0%); C₂₀-C₂₈, 11.7%. Then the residual reaction mixture is neutralized by a solution of 5% aqueous hydrochloric acid in ethanol. A white waxy polymer is obtained, the polymerization activity thereof being 6.21×10⁴ g·mol⁻¹·h⁻¹. The result is shown in Table 1.

It can be seen from Table 1 that, with the catalyst composition comprising 2-imino-1,10-phenanthroline coordinated iron (II) chloride as main catalyst and triethylaluminum as cocatalyst used in the ethylene oligomerization, the catalyst activity is low even with a high amount of cocatalyst (the molar ratio of Al/Fe is 500 or 1000); however, when the amount of cocatalyst is low, oligomerization activity can be up to 2×10⁶ g·mol⁻¹·h⁻¹, which is close to the oligomerization activity when methylaluminoxane is used as cocatalyst at a similar amount (the molar ratio of Al/Fe is 195), and the alpha olefin selectivity is also high. It is illustrated that, when the low cost triethylaluminum is used as cocatalyst, the catalytic activity is unexpectedly appropriate with a low amount of cocatalyst. Moreover, the oligomerization activity increases with the increase of the Al/Fe ratio when the Al/Fe ratio ranges from 30 to less than 200, but decreases with the increase of the Al/Fe ratio when the Al/Fe ratio region is between 200 and 1000.

### Example 15

The ethylene oligomerization reaction is carried out using the complex prepared in Example 1 as the main catalyst and triethylaluminum as the cocatalyst. The process for ethylene oligomerization is as follows: toluene, a 1.21 ml solution (0.74 mol/l) of triethylaluminum (0.8954 mmol) in toluene and a 12 ml solution of 2-acetyl-1,10-phenanthroline (2,6-diethylanil) FeCl₂(3 μmol) in toluene are added into a 300 ml stainless steel autoclave, the total volume being 100 ml and Al/Fe=298.5. When the temperature of reactor is cooled to −15° C., ethylene is added into the autoclave; with the ethylene pressure being kept at 1 MPa and the temperature being kept at −10° C., the reaction is carried out for 30 min under stirring. A small amount of reaction mixture is collected by syringe and neutralized by 5% aqueous hydrogen chloride. The neutralized solution is then analyzed by GC analysis. Result shows that, the oligomerization activity is 5.35×10⁶ g·mol⁻¹(Fe)·h⁻¹, and the contents of oligomers are as follows: C₄, 24.92%; C₆-C₁₀, 57.03%; C₆-C₁₈, 74.09% (in which the content of linear alpha olefins is 98.1%); C₂₀-C₂₈, 0.99%. The residual reaction mixture is neutralized by a solution of 5% aqueous hydrochloric acid in ethanol, no polymer formation being observed. The result is shown in Table 2.

### Example 16

The ethylene oligomerization is carried out using the complex prepared in Example 1 as the main catalyst and triethylaluminum as the cocatalyst. The process for ethylene oligomerization is carried out under the same conditions as in Example 15, except that ethylene is added into the autoclave when the temperature of reactor is cooled to −10° C., and the reaction is carried out for 30 min under stirring with the ethylene pressure being kept at 1 MPa and the temperature being kept at −5° C. A small amount of reaction mixture is collected by syringe and neutralized by 5% aqueous hydrogen chloride. The neutralized solution is then analyzed by GC analysis. Result shows that, the oligomerization activity is 7.74×10⁶ g·mol⁻¹(Fe)·h⁻¹, and the contents of oligomers are as follows: C₄, 26.66%; C₆-C₁₀, 48.32%; C₆-C₁₈, 68.16% (in which the content of linear alpha olefins is 98.4%); C₂₀-C₂₈, 5.18%. Then the residual reaction mixture is neutralized by a solution of 5% aqueous hydrochloric acid in ethanol. A white waxy polymer is obtained, and the polymerization activity is 9.2×10³ g·mol⁻¹(Fe)·h⁻¹. The result is shown in Table 2.

### Example 17

The ethylene oligomerization reaction is carried out using the complex prepared in Example 1 as the main catalyst and triethylaluminum as the cocatalyst. The process for ethylene oligomerization is carried out under the same conditions as in Example 15, except that, ethylene is added into the autoclave when the temperature of reactor is cooled to −5° C., and the reaction is carried out for 30 min under stirring with the ethylene pressure being kept at 1 MPa and the temperature being kept at 0° C. A small amount of reaction mixture is collected by syringe and neutralized by 5% aqueous hydrogen chloride. The neutralized solution is then analyzed by GC analysis. Result shows that, the oligomerization activity is 7.92×10⁶ g·mol⁻¹(Fe)·h⁻¹, and the contents of oligomers are as follows: C₄, 20.60%; C₆-C₁₀, 48.4%; C₆-C₁₈, 75.03% (in which the content of linear alpha olefins is 98.3%); C₂₀-C₂₈, 4.37%. Then the residual reaction mixture is neutralized by a solution of 5% aqueous hydrochloric acid in ethanol. A white waxy polymer is obtained, and the polymerization activity is 2.4×10⁴ g·mol⁻¹(Fe)·h⁻¹. The result is shown in Table 2.

### Example 18

The ethylene oligomerization reaction is carried out using the complex prepared in Example 1 as the main catalyst and triethylaluminum as the cocatalyst. The process for ethylene oligomerization is carried out under the same conditions as in Example 15, except that, ethylene is added into the autoclave when the temperature of reactor is cooled to 2° C., and the reaction is carried out for 30 min under stirring with the ethylene pressure being kept at 1 MPa and the temperature being kept at 5° C. A small amount of reaction mixture is collected by syringe and neutralized by 5% aqueous hydrogen chloride. The neutralized solution is then analyzed by GC analysis. Result shows that, the oligomerization activity is 10.24×10⁶ g·mol⁻¹(Fe)·h⁻¹, and the contents of oligomers are as follows: C₄, 20.43%; C₆-C₁₀, 45.12%; C₆-C₁₈, 69.81% (in which the content of linear alpha olefins is 98.1%); C₂₀-C₂₈, 9.76%. Then the residual reaction mixture is neutralized by a solution of 5% aqueous hydrochloric acid in ethanol, a white waxy polymer is obtained, and polymerization activity is 9.6×10⁴ g·mol⁻¹(Fe)·h⁻¹. The result is shown in Table 2.

### Example 19

The ethylene oligomerization reaction is carried out using the complex prepared in Example 1 as the main catalyst and triethylaluminum as the cocatalyst. The process for ethylene oligomerization is carried out under the same conditions as in Example 15, except that, ethylene is added into the autoclave when the temperature of reactor is cooled to 5° C. and the reaction is carried out for 30 min under stirring with the ethylene pressure being kept at 1 MPa and the temperature being kept at 10° C. A small amount of reaction mixture is collected by syringe and neutralized by 5% aqueous hydrogen chloride. The neutralized solution is then analyzed by GC analysis. Result shows that, the oligomerization activity is 9.35×10⁶ g·mol⁻¹(Fe)·h⁻¹, and the contents of oligomers are as follows: C₄, 19.50%; C₆-C₁₀, 44.13%; C₆-C₁₈, 69.52% (in which the content of linear alpha olefins is 98.3%); C₂₀-C₂₈, 10.98%. Then the residual reaction mixture is neutralized by a solution of 5% aqueous hydrochloric acid in ethanol. A white waxy polymer is obtained, and the polymerization activity is 6.8×10⁴ g·mol⁻¹(Fe)·h⁻¹. The result is shown in Table 2.

### Example 20

The ethylene oligomerization reaction is carried out using the complex prepared in Example 1 as the main catalyst and triethylaluminum as the cocatalyst. The process for ethylene oligomerization is carried out under the same conditions as in Example 15, except that, ethylene is added into the autoclave when the temperature of reactor is cooled to 10° C., and the reaction is carried out for 30 min under stirring with the ethylene pressure being kept at 1 MPa and the temperature being kept at 15° C. A small amount of reaction mixture is collected by syringe and neutralized by 5% aqueous hydrogen chloride. The neutralized solution is then analyzed by GC analysis. Result shows that, the oligomerization activity is 6.88×10⁶ g·mol⁻¹(Fe)·h⁻¹, and the contents of oligomers are as follows: C₄, 20.23%; C₆-C₁₀, 49.23%; C₆-C₁₈, 72.75% (in which the content of linear alpha olefins is 97.7%); C₂₀-C₂₈, 7.02%. Then the residual reaction mixture is neutralized by a solution of 5% aqueous hydrochloric acid in ethanol. A white waxy polymer is obtained, and the polymerization activity is 2.1×10⁴ g·mol⁻¹(Fe)·h⁻¹. The result is shown in Table 2.

### Example 21

The ethylene oligomerization reaction is carried out using the complex prepared in Example 1 as the main catalyst and triethylaluminum as the cocatalyst. The process for ethylene oligomerization is carried out under the same conditions as in Example 15, except that ethylene is added into the autoclave when the temperature of reactor is cooled to 15° C., and the reaction is carried out for 30 min under stirring with the ethylene pressure being kept at 1 MPa and the temperature being kept at 19° C. A small amount of reaction mixture is collected by syringe and neutralized by 5% aqueous hydrogen chloride. The neutralized solution is then analyzed by GC analysis. Result shows that, the oligomerization activity is 5.53×10⁶ g·mol⁻¹(Fe)·h⁻¹, and the contents of oligomers are as follows: C₄, 20.60%; C₆-C₁₀, 48.49%; C₆-C₁₈, 72.21% (in which the content of linear alpha olefins is 98.2%); C₂₀-C₂₈, 7.19%. Then the residual reaction mixture is neutralized by a solution of 5% aqueous hydrochloric acid in ethanol. A white waxy polymer is obtained, and the polymerization activity is 1.4×10⁴ g·mol⁻¹(Fe)·h⁻¹. The result is shown in Table 2.

### Example 22

The ethylene oligomerization reaction is carried out using the complex prepared in Example 1 as the main catalyst and triethylaluminum as the cocatalyst. The process for ethylene oligomerization is carried out under the same conditions as in Example 15, except that, the amount of the solution of triethylaluminum in toluene is 1.62 ml (1.1988 mmol) and Al/Fe=399.6; ethylene is added into the autoclave when the temperature of reactor is cooled to 0° C., and the reaction is carried out for 30 min under stirring with the ethylene pressure being kept at 1 MPa and the temperature being kept at 5° C. A small amount of reaction mixture is collected by syringe and neutralized by 5% aqueous hydrogen chloride. The neutralized solution is then analyzed by GC analysis. Result shows that, the oligomerization activity is 7.18×10⁶ g·mol⁻¹(Fe)·h⁻¹, and the contents of oligomers are as follows: C₄, 20.24%; C₆-C₁₀, 46.56%; C₆-C₁₈, 71.52% (in which the content of linear alpha olefins is 98.1%); C₂₀-C₂₈, 8.23%. Then the residual reaction mixture is neutralized by a solution of 5% aqueous hydrochloric acid in ethanol. A white waxy polymer is obtained, and the polymerization activity is 2.7×10⁴ g·mol⁻¹(Fe)·h⁻¹. The result is shown in Table 2.

### Example 23

The ethylene oligomerization is carried out using the complex prepared in Example 1 as the main catalyst and triethylaluminum as the cocatalyst. The process for ethylene oligomerization is carried out under the same conditions as in Example 15, except that, the amount of the solution of triethylaluminum in toluene is 0.81 ml (0.5994 mmol) and Al/Fe=199.8; ethylene is added into the autoclave when the temperature of reactor is cooled to 0° C., and the reaction is carried out for 30 min under stirring with the ethylene pressure being kept at 1 MPa and the temperature being kept at 5° C. A small amount of reaction mixture is collected by syringe and neutralized by 5% aqueous hydrogen chloride. The neutralized solution is then analyzed by GC analysis. Result shows that, the oligomerization activity is 8.96×10⁶ g·mol⁻¹(Fe)·h⁻¹, and the contents of oligomers are as follows: C₄, 20.02%; C₆-C₁₀, 45.88%; C₆-C₁₈, 70.09% (in which the content of linear alpha olefins is 98.3%); C₂₀-C₂₈, 9.88%. Then the residual reaction mixture is neutralized by a solution of 5% aqueous hydrochloric acid in ethanol. A white waxy polymer is obtained, and the polymerization activity is 3.8×10⁴ g·mol⁻¹(Fe)·h⁻¹. The result is shown in Table 2.

### Example 24

The ethylene oligomerization is carried out using the complex prepared in Example 1 as the main catalyst and triethylaluminum as the cocatalyst. The process for ethylene oligomerization is carried out under the same conditions as in Example 15, except that, the amount of the solution of triethylaluminum in toluene is 0.40 ml (0.296 mmol) and Al/Fe=98.7; ethylene is added into the autoclave when the temperature of reactor is cooled to 0° C., and the reaction is carried out for 30 min under stirring with the ethylene pressure being kept at 1 MPa and the temperature being kept at 5° C. A small amount of reaction mixture is collected by syringe and neutralized by 5% aqueous hydrogen chloride. The neutralized solution is then analyzed by GC analysis. Result shows that, the oligomerization activity is 8.26×10⁶ g·mol⁻¹(Fe)·h⁻¹, and the contents of oligomers are as follows: C₄, 23.56%; C₆-C₁₀, 47.31%; C₆-C₁₈, 69.32% (in which the content of linear alpha olefins is 98.5%); C₂₀-C₂₈, 7.12%. Then the residual reaction mixture is neutralized by a solution of 5% aqueous hydrochloric acid in ethanol. A white waxy polymer is obtained, and the polymerization activity is 7.8×10⁴ g·mol⁻¹(Fe)·h⁻¹. The result is shown in Table 2.

### Example 25

The ethylene oligomerization reaction is carried out using the complex prepared in Example 1 as the main catalyst and triethylaluminum as the cocatalyst. The process for ethylene oligomerization is carried out under the same conditions as in Example 15, except that, the amount of the solution of triethylaluminum in toluene is 0.20 ml (0.148 mmol) and Al/Fe=49.3; ethylene is added into the autoclave when the temperature of reactor is cooled to 0° C., and the reaction is carried out for 30 min under stirring with the ethylene pressure being kept at 1 MPa and the temperature being kept at 5° C. A small amount of reaction mixture is collected by syringe and neutralized by 5% aqueous hydrogen chloride. The neutralized solution is then analyzed by GC analysis. Result shows that, the oligomerization activity is 5.81×10⁶ g·mol⁻¹(Fe)·h⁻¹, and the contents of oligomers are as follows: C₄, 21.95%; C₆-C₁₀, 43.78%; C₆-C₁₈, 68.15% (in which the content of linear alpha olefins is 98.8%); C₂₀-C₂₈, 9.89%. Then the residual reaction mixture is neutralized by a solution of 5% aqueous hydrochloric acid in ethanol. A white waxy polymer is obtained, and the polymerization activity is 5.7×10⁴ g·mol⁻¹(Fe)·h⁻¹. The result is shown in Table 2.

### Example 26

The ethylene oligomerization reaction is carried out using the complex prepared in Example 1 as the main catalyst and triethylaluminum as the cocatalyst. The process for ethylene oligomerization is carried out under the same conditions as in Example 15, except that, ethylene is added into the autoclave when the temperature of reactor is cooled to 2° C., and the reaction is carried out for 30 min under stirring with the ethylene pressure being kept at 2 MPa and the temperature being kept at 5° C. A small amount of reaction mixture is collected by syringe and neutralized by 5% aqueous hydrogen chloride. The neutralized solution is then analyzed by GC analysis. Result shows that, the oligomerization activity is 11.31×10⁶ g·mol⁻¹(Fe)·h⁻¹, and the contents of oligomers are as follows: C₄, 21.53%; C₆-C₁₀, 44.57%; C₆-C₁₈, 69.26% (in which the content of linear alpha olefins is 98.3%); C₂₀-C₂₈, 9.21%. Then the residual reaction mixture is neutralized by a solution of 5% aqueous hydrochloric acid in ethanol. A white waxy polymer is obtained, and the polymerization activity is 9.8×10⁴ g·mol⁻¹(Fe)·h⁻¹. The result is shown in Table 2.

### Example 27

The ethylene oligomerization is carried out using the complex prepared in Example 1 as the main catalyst and triethylaluminum as the cocatalyst. The process for ethylene oligomerization is carried out under the same conditions as in Example 15, except that, ethylene is added into the autoclave when the temperature of reactor is cooled to 2° C., and the reaction is carried out for 30 min under stirring with the ethylene pressure being kept at 3 MPa and the temperature being kept at 5° C. A small amount of reaction mixture is collected by syringe and neutralized by 5% aqueous hydrogen chloride. The neutralized solution is then analyzed by GC analysis. Result shows that, the oligomerization activity is 13.54×10⁶ g·mol⁻¹(Fe)·h⁻¹, and the contents of oligomers are as follows: C₄, 22.12%; C₆-C₁₀, 44.43%; C₆-C₁₈, 69.12% (in which the content of linear alpha olefins is 98.2%); C₂₀-C₂₈, 8.76%. Then the residual reaction mixture is neutralized by a solution of 5% aqueous hydrochloric acid in ethanol. A white waxy polymer is obtained, and the polymerization activity is 1.0×10⁵ g·mol⁻¹(Fe)·h⁻¹. The result is shown in Table 2.

### Comparative Example 5

The process for ethylene oligomerization is repeated as in Example 23, except that: ethylene is added into the autoclave when the temperature reaches 40° C., and the reaction is carried out for 30 min under stirring with the ethylene pressure being kept at 1 MPa and the temperature being kept at 40° C. A small amount of reaction mixture is collected by syringe and neutralized by 5% aqueous hydrogen chloride. The neutralized solution is then analyzed by GC analysis. Result shows that, the oligomerization activity is 2.12×10⁶ g·mol⁻¹(Fe)·h⁻¹, and the contents of oligomers are as follows: C₄, 13.1%; C₆-C₁₀, 64.0%; C₆-C₁₈, 82.8% (in which the content of linear alpha olefins is 98.2%); C₂₀-C₂₈, 4.1%. The residual reaction mixture is neutralized by a solution of 5% aqueous hydrochloric acid in ethanol, no polymer formation being observed. The result is shown in Table 2.

### Comparative Example 6

The process for ethylene oligomerization is repeated as in Example 15, except that, ethylene is added into the autoclave when the temperature reaches 40° C., and the reaction is carried out for 30 min under stirring with the ethylene pressure being kept at 1 MPa and the temperature being kept at 40° C. A small amount of reaction mixture is collected by syringe and neutralized by 5% aqueous hydrogen chloride. The neutralized solution is then analyzed by GC analysis. Result shows that, the oligomerization activity is 1.93×10⁶ g·mol⁻¹(Fe)·h⁻¹, and the contents of oligomers are as follows: C₄, 20.61%; C₆-C₁₀, 55.17%; C₆-C₁₈, 75.37% (in which the content of linear alpha olefins is 97.0%); C₂₀-C₂₈, 4.02%. The residual reaction mixture is neutralized by a solution of 5% aqueous hydrochloric acid in ethanol, no polymer formation being observed. The result is shown in Table 2.

### Comparative Example 7

The ethylene oligomerization reaction is carried out using the complex prepared in Example 1 as the main catalyst and the process in Example 1. The differences of Comparative Example 7 from Example 1 lie in that, methylaluminoxane is used as cocatalyst, the amount of the solution of methylaluminoxane in toluene being 0.54 ml (1.5 mol/l) and Al/Fe 400. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 1.08×10⁷ g·mol⁻¹(Fe)·h¹, and the contents of oligomers are as follows: C₄, 16.4%; C₆-C₁₀, 45.2%; C₆-C₁₈, 73.0% (in which the content of linear alpha olefins is 95.0%); C₂₀-C₂₈, 10.6%. Then the residual reaction mixture is neutralized by a solution of 5% aqueous hydrochloric acid in ethanol. A white waxy polymer is obtained, and the polymerization activity is 4.65×10⁵ g·mol⁻¹(Fe)·h⁻¹. The result is shown in Table 2.

### Comparative Example 8

The ethylene oligomerization reaction is carried out using the complex prepared in Example 1 as main catalyst and the process in Example 1. The differences of Comparative Example 8 from Example 1 lie in that, methylaluminoxane is used as cocatalyst, the amount of the solution of methylaluminoxane in toluene being 1.36 ml (1.5 mol/l) and Al/Fe being 1000. With the ethylene pressure being kept at 1 MPa, the reaction is carried out at 40° C. for 30 min under stirring. A small amount of reaction mixture is collected by syringe and neutralized by 5% aqueous hydrogen chloride. The neutralized solution is then analyzed by GC analysis. Result shows that, the oligomerization activity is 1.41×10⁷ g·mol⁻¹(Fe)·h⁻¹, and the contents of oligomers are as follows: C₄, 35.0%; C₆-C₁₀, 40.4%; C₆-C₁₈, 64.7% (in which the content of linear alpha olefins is 99.3%); C₂₀-C₂₈, 0.3%. Then the residual reaction mixture is neutralized by a solution of 5% aqueous hydrochloric acid in ethanol. A white waxy polymer is obtained, and the polymerization activity is 4.23×10⁵ g·mol⁻¹(Fe)·h⁻¹. The result is shown in Table 2.

It can be seen from Table 2 that, when the catalyst composition comprising 2-imino-1,10-phenanthroline coordinated iron (II) chloride as main catalyst and triethylaluminum as cocatalyst is used in the ethylene oligomerization, the catalytic activity is high at a low temperature (−10 to 19° C.), and the oligomerization activity can be in excess of 10⁷ g·mol⁻¹·h⁻¹, which is several to dozens times higher than that at 40° C., and even close to the oligomerization activity when methylaluminoxane is used as cocatalyst at the temperature of 40° C. at which the oligomerization activity is the highest. It means that according to the present invention, when the low cost triethylaluminum is used as cocatalyst, the catalyst activity can be unexpectedly high at a low temperature. Moreover, in the temperature range of from −10 to 19° C., the oligomerization activity increases initially and then decreases with the increase of the temperature, and the highest value of oligomerization activity is at 5° C.

TABLE 1

| | | | | | | | | | C6-C18 | | |
|---|---|---|---|---|---|---|---|---|---|---|---|
| | | | | | | oligomerization | | | | linear | |
| | | P | T | central | Al/central | activity | C4 | C6-C10 | | α-olefin | C20-C28 |
| Num. | cocatalyst | (MPa) | (° C.) | metal | metal | (10⁶ g · mol⁻¹ · h⁻¹) | (%) | (%) | (%) | (%) | (%) |
| Example 1 | AlEt₃ | 1 | 40 | Fe | 196 | 2.02 | 12.0 | 64.7 | 87.0 | 98.0 | 1.0 |
| Example 2 | AlEt₃ | 1 | 40 | Fe | 199.8 | 2.02 | 12.1 | 64.5 | 86.8 | 97.5 | 1.1 |
| Example 3 | AlEt₃ | 1 | 40 | Fe | 189 | 1.98 | 11.6 | 64.8 | 86.9 | 98.0 | 1.5 |
| Example 4 | AlEt₃ | 1 | 40 | Fe | 178 | 1.98 | 10.5 | 65.1 | 87.7 | 98.3 | 1.8 |
| Example 5 | AlEt₃ | 1 | 40 | Fe | 148 | 1.21 | 24.7 | 57.4 | 72.7 | 92.9 | 2.6 |
| Example 6 | AlEt₃ | 1 | 40 | Fe | 101 | 1.01 | 21.6 | 53.6 | 75.3 | 89.9 | 3.1 |
| Example 7 | AlEt₃ | 1 | 40 | Fe | 50 | 0.12 | 7.4 | 86.8 | 92.6 | 92.5 | 0 |
| Example 8 | AlEt₃ | 1 | 40 | Fe | 30 | 0.08 | 6.9 | 87.1 | 93.1 | 91.5 | 0 |
| Example 9 | AlEt₃ | 1 | 40 | Co | 196 | 1.51 | 100 | — | — | — | — |
| Example 10 | AlEt₃ | 1 | 40 | Ni | 196 | 1.40 | 100 | — | — | — | — |
| Example 11 | AlEt₃ | 2 | 40 | Fe | 196 | 3.21 | 19.40 | 53.02 | 75.68 | 96.9 | 4.92 |
| Example 12 | AlEt₃ | 2 | 40 | Fe | 199.8 | 3.83 | 21.05 | 52.37 | 73.36 | 97.5 | 5.59 |
| Example 13 | AlEt₃ | 3 | 40 | Fe | 196 | 6.40 | 17.5 | 46.2 | 71.5 | 98.7 | 11.0 |
| Example 14 | AlEt₃ | 3 | 40 | Fe | 148 | 5.21 | 19.5 | 53.4 | 75.8 | 98.4 | 4.7 |
| Comparative | AlEt₃ | 1 | 40 | Fe | 500 | 0.88 | 37.0 | 52.0 | 63.0 | 91.5 | 0 |
| Example1 | | | | | | | | | | | |
| Comparative | AlEt₃ | 1 | 40 | Fe | 500 | 0.271 | 39.3 | C6 29.3 | | C8~C22 31.4% | |
| Example2 | | | | | | | | | | | |
| Comparative | AlEt₃ | 1 | 40 | Fe | 1000 | 0.18 | 43.9 | 50.9 | 55.5 | 84.3 | 0.6 |
| Example3 | | | | | | | | | | | |
| Comparative | MAO | 1 | 40 | Fe | 195 | 2.50 | 14.2 | 44.9 | 74.1 | 89.0 | 11.7 |
| Example4 | | | | | | | | | | | |

TABLE 2

| | | | | | | | | C₆-C₁₈ | | | |
|---|---|---|---|---|---|---|---|---|---|---|---|
| | | | | Al/ | oligomerization | | | | linear | | polymerization |
| | | P | T | central | activity | C₄ | C₆-C₁₀ | | α-olefin | C₂₀-C₂₈ | activity |
| Num. | cocatalyst | (MPa) | (° C.) | metal | (10⁶ g · mol⁻¹ · h⁻¹) | (%) | (%) | (%) | (%) | (%) | (10⁴ g · mol⁻¹ · h⁻¹) |
| Example 15 | AlEt₃ | 1 | −10 | 298.5 | 5.35 | 24.92 | 57.03 | 74.09 | 98.1 | 0.99 | — |
| Example 16 | AlEt₃ | 1 | −5 | 298.5 | 7.74 | 26.66 | 48.32 | 68.16 | 98.4 | 5.18 | 0.92 |
| Example 17 | AlEt₃ | 1 | 0 | 298.5 | 7.92 | 20.60 | 48.40 | 75.03 | 98.3 | 4.37 | 2.4 |
| Example 18 | AlEt₃ | 1 | 5 | 298.5 | 10.24 | 20.43 | 45.12 | 69.81 | 98.1 | 9.76 | 9.6 |
| Example 19 | AlEt₃ | 1 | 10 | 298.5 | 9.35 | 19.50 | 44.13 | 69.52 | 98.3 | 10.98 | 6.8 |
| Example 20 | AlEt₃ | 1 | 15 | 298.5 | 6.88 | 20.23 | 49.23 | 72.75 | 97.7 | 7.02 | 2.1 |
| Example 21 | AlEt₃ | 1 | 19 | 298.5 | 5.53 | 20.60 | 48.49 | 72.21 | 98.2 | 7.19 | 1.4 |
| Example 22 | AlEt₃ | 1 | 5 | 399.6 | 7.18 | 20.24 | 46.56 | 71.52 | 98.1 | 8.23 | 2.7 |
| Example 23 | AlEt₃ | 1 | 5 | 199.8 | 8.96 | 20.02 | 45.88 | 70.09 | 98.3 | 9.88 | 3.8 |
| Example 24 | AlEt₃ | 1 | 5 | 98.7 | 8.26 | 23.56 | 47.31 | 69.32 | 98.5 | 7.12 | 7.8 |
| Example 25 | AlEt₃ | 1 | 5 | 49.3 | 5.81 | 21.95 | 43.78 | 68.15 | 98.8 | 9.89 | 5.7 |
| Example 26 | AlEt₃ | 2 | 5 | 298.5 | 11.31 | 21.53 | 44.57 | 69.26 | 98.3 | 9.21 | 9.8 |
| Example 27 | AlEt₃ | 3 | 5 | 298.5 | 13.54 | 22.12 | 44.43 | 69.12 | 98.2 | 8.76 | 10.0 |
| Comparative | AlEt₃ | 1 | 40 | 199.8 | 2.12 | 13.1 | 64.0 | 82.8 | 98.2 | 4.1 | 0.68 |
| Example 5 | | | | | | | | | | | |
| Comparative | AlEt₃ | 1 | 40 | 298.5 | 1.93 | 20.61 | 55.17 | 75.37 | 97.0 | 4.02 | 0.57 |
| Example 6 | | | | | | | | | | | |
| Comparative | AlEt₃ | 1 | 40 | 500 | 0.88 | 37.0 | 52.0 | 63.0 | 91.5 | 0 | — |
| Example 1 | | | | | | | | | | | |
| Comparative | AlEt₃ | 1 | 40 | 500 | 0.271 | 39.3 | C₆ 29.3 | | C₈~C₂₂ 31.4% | | — |
| Example 2 | | | | | | | | | | | |
| Comparative | AlEt₃ | 1 | 40 | 1000 | 0.18 | 43.9 | 50.9 | 55.5 | 84.3 | 0.6 | — |
| Example 3 | | | | | | | | | | | |
| Comparative | MAO | 1 | 40 | 195 | 2.50 | 14.2 | 44.9 | 74.1 | 89.0 | 11.7 | 6.21 |
| Example 4 | | | | | | | | | | | |
| Comparative | MAO | 1 | 40 | 400 | 10.8 | 16.4 | 45.2 | 73.0 | 95.0 | 10.6 | 46.5 |
| Example 7 | | | | | | | | | | | |
| Comparative | MAO | 1 | 40 | 1000 | 14.1 | 35.0 | 40.4 | 64.7 | 99.3 | 0.3 | 42.3 |
| Example 8 | | | | | | | | | | | |

1. A catalyst composition for ethylene oligomerization, comprising 2-imino-1,10-phenanthroline coordinated iron (II), cobalt (II) or nickel (II) chloride as shown in Formula (I) as the main catalyst and triethylaluminum as the cocatalyst, wherein the molar ratio of aluminum in the cocatalyst to the central metal in the main catalyst ranges from 100 to less than 200 or from 30 to 50: 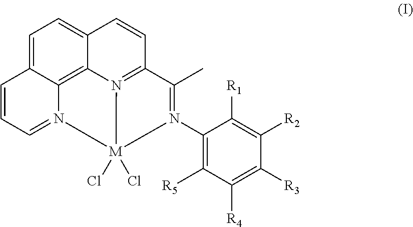
wherein M is the central metal, selected from Fe²⁺, Co²⁺ and Ni²⁺; and R₁-R₅ are independently selected from hydrogen, (C₁-C₆) alkyl, halogen, (C₁-C₆) alkoxyl and nitro group.

2. The catalyst composition according to claim 1, wherein the molar ratio of aluminum in the cocatalyst to the central metal in the main catalyst ranges selected from the group consisting of from 100 to 199.8, from 148 to 196, and from 178 to 196.

3-5. (canceled)

6. The catalyst composition according to claim 1, wherein R₁-R₅ in the main catalyst are independently selected from hydrogen, methyl, ethyl, isopropyl, fluoro, chloro, bromo, methoxyl, ethoxyl and nitro group, wherein R₁ and R₅ in the main catalyst are ethyl groups, and R₂-R₄ in the main catalyst are hydrogen atoms.

7. (canceled)

8. The catalyst composition according to claim 1, wherein M and R₁-R₅ in the main catalyst are defined as follows:
1: M=Fe²⁺, R₁=Me, R₂═R₃═R₄═R₅═H;
2: M=Fe²⁺, R₂=Me, R₁═R₃═R₄═R₅═H;
3: M=Fe²⁺, R₃=Me, R₁═R₂═R₄═R₅═H;
4: M=Fe²⁺, R₁═R₂=Me, R₃═R₄═R₅═H;
5: M=Fe²⁺, R₁═R₃=Me, R₂═R₄═R₅═H;
6: M=Fe²⁺, R₁═R₄=Me, R₂═R₃═R₅═H;
7: M=Fe²⁺, R₁═R₅=Me, R₂═R₃═R₄═H;
8: M=Fe²⁺, R₂═R₃=Me, R₁═R₄═R₅═H;
9: M=Fe²⁺, R₂═R₄=Me, R₁═R₃═R₅═H;
10: M=Fe²⁺, R₁═R₃═R₅=Me, R₂═R₄═H;
11: M=Fe²⁺, R₁=Et, R₂═R₃═R₄═R₅═H;
12: M=Fe²⁺, R₁=Et, R₅=Me, R₂═R₃═R₄═H;
13: M=Fe²⁺, R₁═R₅=Et, R₂═R₃═R₄═H;
14: M=Fe²⁺, R₁=iPr, R₂═R₃═R₄═R₅═H;
15: M=Fe²⁺, R₁═R₅=iPr, R₂═R₃═R₄═H;
16: M=Co²⁺, R₁=Me, R₂═R₃═R₄═R₅═H;
17: M=Co²⁺, R₂=Me, R₁═R₃═R₄═R₅═H;
18: M=Co²⁺, R₃=Me, R₁═R₂═R₄═R₅═H;
19: M=Co²⁺, R₁═R₂=Me, R₃═R₄═R₅═H;
20: M=Co²⁺, R₁═R₃=Me, R₂═R₄═R₅═H;
21: M=Co²⁺, R₁═R₄=Me, R₂═R₃═R₅═H;
22: M=Co²⁺, R₁═R₅=Me, R₂═R₃═R₄═H;
23: M=Co²⁺, R₂═R₃=Me, R₁═R₄═R₅═H;
24: M=Co²⁺, R₂═R₄=Me, R₁═R₃═R₅═H;
25: M=Co²⁺, R₁═R₃═R₅=Me, R₂═R₄═H;
26: M=Co²⁺, R₁=Et, R₂═R₃═R₄═R₅═H;
27: M=Co²⁺, R₁=Et, R₅=Me, R₂═R₃═R₄═H;
28: M=Co²⁺, R₁═R₅=Et, R₂═R₃═R₄═H;
29: M=Co²⁺, R₁=iPr, R₂═R₃═R₄═R₅═H;
30: M=Co²⁺, R₁═R₅=iPr, R₂═R₃═R₄═H;
31: M=Ni²⁺, R₁=Me, R₂═R₃═R₄═R₅═H;
32: M=Ni²⁺, R₂=Me, R₁═R₃═R₄═R₅═H;
33: M=Ni²⁺, R₃=Me, R₁═R₂═R₄═R₅═H;
34: M=Ni²⁺, R₁═R₂=Me, R₃═R₄═R₅═H;
35: M=Ni²⁺, R₁═R₃=Me, R₂═R₄═R₅═H;
36: M=Ni²⁺, R₁═R₄=Me, R₂═R₃═R₅═H;
37: M=Ni²⁺, R₁═R₅=Me, R₂═R₃═R₄═H;
38: M=Ni²⁺, R₂═R₃=Me, R₁═R₄═R₅═H;
39: M=Ni²⁺, R₂═R₄=Me, R₁═R₃═R₅═H;
40: M=Ni²⁺, R₁═R₃═R₅=Me, R₂═R₄═H;
41: M=Ni²⁺, R₁=Et, R₂═R₃═R₄═R₅═H;
42: M=Ni²⁺, R₁=Et, R₅=Me, R₂═R₃═R₄═H;
43: M=Ni²⁺, R₁═R₅=Et, R₂═R₃═R₄═H;
44: M=Ni²⁺, R₁=iPr, R₂═R₃═R₄═R₅═H;
45: M=Ni²⁺, R₁═R₅=iPr, R₂═R₃═R₄═H.

9. A Process for ethylene oligomerization, wherein a catalyst composition comprising 2-imino-1,10-phenanthroline coordinated iron (II), cobalt (II) or nickel (II) chloride as shown in Formula (I) as the main catalyst and triethylaluminum as the cocatalyst is used, and the molar ratio of aluminum in the cocatalyst to the central metal in the main catalyst ranges from 100 to less than 200 or from 30 to 50: 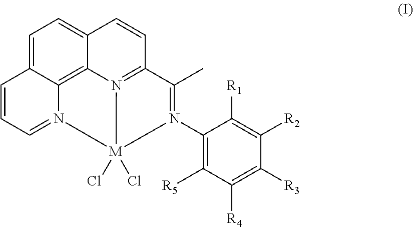
wherein M is the central metal, selected from Fe²⁺, Co²⁺ and Ni²⁺; and R₁-R₅ are independently selected from hydrogen, (C₁-C₆) alkyl, halogen, (C₁-C₆) alkoxyl and nitro group.

10. The process according to claim 9, wherein the molar ratio of aluminum in the cocatalyst to the central metal in the main catalyst ranges are selected from the group consisting of from 100 to 199.8, from 148 to 196, and from 178 to 196.

11-13. (canceled)

14. The process according to claim 9, wherein R₁-R₅ in the main catalyst are independently selected from hydrogen, methyl, ethyl, isopropyl, fluoro, chloro, bromo, methoxyl, ethoxyl and nitro group, wherein R₁ and R₅ in the main catalyst are ethyl groups, and R₂-R₄ in the main catalyst are hydrogen atoms.

15. (canceled)

16. The process according to claim 9, wherein M and R₁-R₅ in the main catalyst are defined as follows:
1: M=Fe²⁺, R₁=Me, R₂═R₃═R₄═R₅═H;
2: M=Fe²⁺, R₂=Me, R₁═R₃═R₄═R₅═H;
3: M=Fe²⁺, R₃=Me, R₁═R₂═R₄═R₅═H;
4: M=Fe²⁺, R₁═R₂=Me, R₃═R₄═R₅═H;
5: M=Fe²⁺, R₁═R₃=Me, R₂═R₄═R₅═H;
6: M=Fe²⁺, R₁═R₄=Me, R₂═R₃═R₅═H;
7: M=Fe²⁺, R₁═R₅=Me, R₂═R₃═R₄═H;
8: M=Fe²⁺, R₂═R₃=Me, R₁═R₄═R₅═H;
9: M=Fe²⁺, R₂═R₄=Me, R₁═R₃═R₅═H;
10: M=Fe²⁺, R₁═R₃═R₅=Me, R₂═R₄═H;
11: M=Fe²⁺, R₁=Et, R₂═R₃═R₄═R₅═H;
12: M=Fe²⁺, R₁=Et, R₅=Me, R₂═R₃═R₄═H;
13: M=Fe²⁺, R₁═R₅=Et, R₂═R₃═R₄═H;
14: M=Fe²⁺, R₁=iPr, R₂═R₃═R₄═R₅═H;
15: M=Fe²⁺, R₁═R₅=iPr, R₂═R₃═R₄═H;
16: M=Co²⁺, R₁=Me, R₂═R₃═R₄═R₅═H;
17: M=Co²⁺, R₂=Me, R₁═R₃═R₄═R₅═H;
18: M=Co²⁺, R₃=Me, R₁═R₂═R₄═R₅═H;
19: M=Co²⁺, R₁═R₂=Me, R₃═R₄═R₅═H;
20: M=Co²⁺, R₁═R₃=Me, R₂═R₄═R₅═H;
21: M=Co²⁺, R₁═R₄=Me, R₂═R₃═R₅═H;
22: M=Co²⁺, R₁═R₅=Me, R₂═R₃═R₄═H;
23: M=Co²⁺, R₂═R₃=Me, R₁═R₄═R₅═H;
24: M=Co²⁺, R₂═R₄=Me, R₁═R₃═R₅═H;
25: M=Co²⁺, R₁═R₃═R₅=Me, R₂═R₄═H;
26: M=Co²⁺, R₁=Et, R₂═R₃═R₄═R₅═H;
27: M=Co²⁺, R₁=Et, R₅=Me, R₂═R₃═R₄═H;
28: M=Co²⁺, R₁═R₅=Et, R₂═R₃═R₄═H;
29: M=Co²⁺, R₁=iPr, R₂═R₃═R₄═R₅═H;
30: M=Co²⁺, R₁═R₅=iPr, R₂═R₃═R₄═H;
31: M=Ni²⁺, R₁=Me, R₂═R₃═R₄═R₅═H;
32: M=Ni²⁺, R₂=Me, R₁═R₃═R₄═R₅═H;
33: M=Ni²⁺, R₃=Me, R₁═R₂═R₄═R₅═H;
34: M=Ni²⁺, R₁═R₂=Me, R₃═R₄═R₅═H;
35: M=Ni²⁺, R₁═R₃=Me, R₂═R₄═R₅═H;
36: M=Ni²⁺, R₁═R₄=Me, R₂═R₃═R₅═H;
37: M=Ni²⁺, R₁═R₅=Me, R₂═R₃═R₄═H;
38: M=Ni²⁺, R₂═R₃=Me, R₁═R₄═R₅═H;
39: M=Ni²⁺, R₂═R₄=Me, R₁═R₃═R₅═H;
40: M=Ni²⁺, R₁═R₃═R₅=Me, R₂═R₄═H;
41: M=Ni²⁺, R₁=Et, R₂═R₃═R₄═R₅═H;
42: M=Ni²⁺, R₁=Et, R₅=Me, R₂═R₃═R₄═H;
43: M=Ni²⁺, R₁═R₅=Et, R₂═R₃═R₄═H;
44: M=Ni²⁺, R₁=iPr, R₂═R₃═R₄═R₅═H;
45: M=Ni²⁺, R₁═R₅=iPr, R₂═R₃═R₄═H.

17. The process according to claim 9, wherein the reaction temperature of ethylene oligomerization is from 20 to 80° C.

18. The process according to claim 9, wherein the reaction pressure of ethylene oligomerization is from 1 to 5 MPa.

19. A process for ethylene oligomerization, wherein a catalyst composition comprising 2-imino-1,10-phenanthroline coordinated iron(II), cobalt(II) or nickel(II) chloride as shown in Formula (I) as the main catalyst and triethylaluminum as cocatalyst is used, and the reaction temperature of ethylene oligomerization is from −10 to 19° C.: 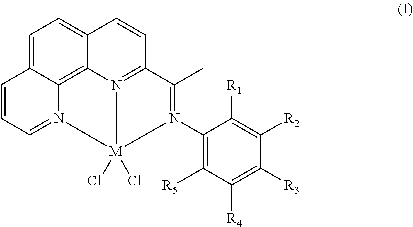
wherein M is the central metal, selected from Fe²⁺, Co²⁺ and Ni²⁺; and R₁-R₅ are independently selected from hydrogen, (C₁-C₆) alkyl, halogen, (C₁-C₆) alkoxyl and nitro group.

20. The process according to claim 19, wherein the reaction temperature of ethylene oligomerization is selected from the group consisting of from −10 to 15° C., from 0 to 15° C., and from 5 to 10° C.

21-22. (canceled)

23. The process according to claim 19, wherein R₁-R₅ in the main catalyst are independently selected from hydrogen, methyl, ethyl, isopropyl, fluoro, chloro, bromo, methoxyl, ethoxyl and nitro group, wherein R₁ and R₅ in the main catalyst are ethyl groups, and R₂-R₄ in the main catalyst are hydrogen atoms.

24. (canceled)

25. The process according to claim 19, wherein M and R₁-R₅ in the main catalyst are defined as follows:
1: M=Fe²⁺, R₁=Me, R₂═R₃═R₄═R₅═H; 2: M=Fe²⁺, R₂=Me, R₁═R₃═R₄═R₅═H;
3: M=Fe²⁺, R₃=Me, R₁═R₂═R₄═R₅═H;
4: M=Fe²⁺, R₁═R₂=Me, R₃═R₄═R₅═H;
5: M=Fe²⁺, R₁═R₃=Me, R₂═R₄═R₅═H;
6: M=Fe²⁺, R₁═R₄=Me, R₂═R₃═R₅═H;
7: M=Fe²⁺, R₁═R₅=Me, R₂═R₃═R₄═H;
8: M=Fe²⁺, R₂═R₃=Me, R₁═R₄═R₅═H;
9: M=Fe²⁺, R₂═R₄=Me, R₁═R₃═R₅═H;
10: M=Fe²⁺, R₁═R₃═R₅=Me, R₂═R₄═H;
11: M=Fe²⁺, R₁=Et, R₂═R₃═R₄═R₅═H;
12: M=Fe²⁺, R₁=Et, R₅=Me, R₂═R₃═R₄═H;
13: M=Fe²⁺, R₁═R₅=Et, R₂═R₃═R₄═H;
14: M=Fe²⁺, R₁=iPr, R₂═R₃═R₄═R₅═H;
15: M=Fe²⁺, R₁═R₅=iPr, R₂═R₃═R₄═H;
16: M=Co²⁺, R₁=Me, R₂═R₃═R₄═R₅═H;
17: M=Co²⁺, R₂=Me, R₁═R₃═R₄═R₅═H;
18: M=Co²⁺, R₃=Me, R₁═R₂═R₄═R₅═H;
19: M=Co²⁺, R₁═R₂=Me, R₃═R₄═R₅═H;
20: M=Co²⁺, R₁═R₃=Me, R₂═R₄═R₅═H;
21: M=Co²⁺, R₁═R₄=Me, R₂═R₃═R₅═H;
22: M=Co²⁺, R₁═R₅=Me, R₂═R₃═R₄═H;
23: M=Co²⁺, R₂═R₃=Me, R₁═R₄═R₅═H;
24: M=Co²⁺, R₂═R₄=Me, R₁═R₃═R₅═H;
25: M=Co²⁺, R₁═R₃═R₅=Me, R₂═R₄═H;
26: M=Co²⁺, R₁=Et, R₂═R₃═R₄═R₅═H;
27: M=Co²⁺, R₁=Et, R₅=Me, R₂═R₃═R₄═H;
28: M=Co²⁺, R₁═R₅=Et, R₂═R₃═R₄═H;
29: M=Co²⁺, R₁=iPr, R₂═R₃═R₄═R₅═H;
30: M=Co²⁺, R₁═R₅=iPr, R₂═R₃═R₄═H;
31: M=Ni²⁺, R₁=Me, R₂═R₃═R₄═R₅═H;
32: M=Ni²⁺, R₂=Me, R₁═R₃═R₄═R₅═H;
33: M=Ni²⁺, R₃=Me, R₁═R₂═R₄═R₅═H;
34: M=Ni²⁺, R₁═R₂=Me, R₃═R₄═R₅═H;
35: M=Ni²⁺, R₁═R₃=Me, R₂═R₄═R₅═H;
36: M=Ni²⁺, R₁═R₄=Me, R₂═R₃═R₅═H;
37: M=Ni²⁺, R₁═R₅=Me, R₂═R₃═R₄═H;
38: M=Ni²⁺, R₂═R₃=Me, R₁═R₄═R₅═H;
39: M=Ni²⁺, R₂═R₄=Me, R₁═R₃═R₅═H;
40: M=Ni²⁺, R₁═R₃═R₅=Me, R₂═R₄═H;
41: M=Ni²⁺, R₁=Et, R₂═R₃═R₄═R₅═H;
42: M=Ni²⁺, R₁=Et, R₅=Me, R₂═R₃═R₄═H;
43: M=Ni²⁺, R₁═R₅=Et, R₂═R₃═R₄═H;
44: M=Ni²⁺, R₁=iPr, R₂═R₃═R₄═R₅═H;
45: M=Ni²⁺, R₁═R₅=iPr, R₂═R₃═R₄═H.

26. The process according to claim 19, wherein the molar ratio of aluminum in the cocatalyst to the central metal in the main catalyst is in ranges selected from the group consisting of from 49 to 500, from 100 to 400, and from 200 to 300.

27-28. (canceled)

29. The process according to claim 26, wherein the molar ratio of aluminum in the cocatalyst to central metal in the main catalyst is 300.

30. The process according to claim 19, wherein the reaction pressure of the ethylene oligomerization is from 0.1 to 30 MPa.

31. The process according to claim 30, wherein the reaction pressure of the ethylene oligomerization is from 1 to 5 MPa.

32. The process according to claim 19, wherein the organic solvent used in the ethylene oligomerization is selected from the group consisting of toluene, cyclohexane, ether, tetrahydrofuran, ethanol, benzene, xylene and dicholomethane.

33. The process according to claim 32, wherein the organic solvent used in the ethylene oligomerization is toluene.
